(19) United States
(12) Patent Application Publication
(10) Pub. No.: US 2026/0315004 A1
(43) Pub. Date: Oct. 15, 2026

(54) POROUS METALLIC MICRONEEDLE DEVICE AND METHOD OF FABRICATION THEREOF

(71) Applicant: City University of Hong Kong, Kowloon (HK)

(72) Inventors: Chenjie Xu, Kowloon Tong (HK); Fang Leng, Kowloon Tong (HK)

(21) Appl. No.: 19/635,977

(22) Filed: Apr. 1, 2026

Related U.S. Application Data
(60) Provisional application No. 63/788,926, filed on Apr. 15, 2025.

(51) Int. Cl. A61B 5/00; A61B 10/00

(52) U.S. Cl. CPC A61B 5/685; A61B 5/4552; A61B 10/0045; A61B 2010/008; A61B 2562/12

(57) ABSTRACT

A method of fabricating a porous metallic microneedle device includes: casting a hydrogel precursor in a mold to form one or more hydrogel microneedles; immersing at least part of the hydrogel microneedles in a solution including metallic ions, to load the hydrogel microneedles with the metal ions; and sintering the metal ion-loaded hydrogel microneedles to form the porous metallic microneedle device.

### TECHNICAL FIELD

Embodiments of the present invention relate to a microneedle device and a method of fabrication thereof. More particularly, embodiments of the present invention relate to a porous metallic microneedle device and its fabrication method.

### BACKGROUND OF THE INVENTION

Periodontal disease is the most common oral disease worldwide, affecting 19% of the global adult population. The collection and analysis of gingival crevicular fluid (GCF) have been a recognized approach to develop an accurate picture of periodontal diseases and the treatment effectiveness. GCF, located in the sulcus/periodontal pocket, is a complex mixture derived from serum and from locally generated mediators of inflammation and tissue turnover/breakdown. Its volume and composition reflect the health of periodontium. Representative periodontal disease related biomarkers in GCF include matrix metalloproteinases (MMP) such as MMP-8 and interleukins (IL) like IL-1β (5).

GCF can be collected through gingival washing techniques, paper strip absorption and microcapillary pipetting while the filter paper (e.g. Perio paper) is most frequently used for its easiness and simplicity. However, attempts to position these paper strips to the known depth of a particular periodontal pocket are typically unsuccessful because the fluid-saturated paper tends to crumple up under the pressure of insertion. Also, paper strips often meet blood and saliva, leading to contamination. Therefore, there is a great need for a new, effective, and contamination-free GCF collection method for periodontal healthcare.

Microneedle devices for fluid extraction can be categorized as follows. The first category comprises hollow microneedles with slender channels. These microneedles typically have one or more such channels and are made of polymers. Using these hollow microneedles for fluid extraction generally requires the application of negative pressure; otherwise, the extraction efficiency is low. They also suffer from inadequate mechanical strength. The second category is silicon microneedles, including silicon and metal microneedles. Silicon-based microneedles are brittle and prone to fracture. Existing porous metal microneedles for fluid extraction are made using sintered titanium particles at 1250° C. in an inert gas environment. Other methods of manufacturing metal microneedles mainly involve electroplating deposition.

The invention seeks to eliminate or at least to mitigate such shortcomings by providing a porous metallic microneedle device and its fabrication method.

### SUMMARY OF THE INVENTION

According to a first aspect of the present invention, there is provided a method of fabricating a porous metallic microneedle device, comprising steps of: casting a hydrogel precursor in a mold to form one or more hydrogel microneedles; immersing at least part of the hydrogel microneedles in a solution comprising metallic ions, to load the hydrogel microneedles with the metal ions; and sintering the metal ion-loaded hydrogel microneedles to form the porous metallic microneedle device.

In an embodiment, the sintering of the metal-loaded hydrogel microneedles is performed at a maximum temperature of about 700° C. in an air atmosphere.

In an embodiment, the metal-loaded hydrogel microneedles are sintered by raising the temperature at 0.25° C./min to the maximum temperature, maintaining at the maximum temperature for a predetermined period, and then cooling at 2° C./min to room temperature.

In an embodiment, wherein the metal-loaded hydrogel microneedles are maintained at the maximum temperature for about 3 hours.

In an embodiment, the hydrogel precursor comprises one or more carboxy group, wherein the hydrogel precursor is selected from a group consisting of hyaluronic acid, collagen, alginate, PLGA, or a combination thereof.

In an embodiment, the metallic ions are silver ions or iron ions.

In an embodiment, the metallic ions are silver ions and the solution is a silver nitrate solution.

In an embodiment, the silver nitrate solution has a concentration ranging from 0.25M to 4M.

In an embodiment, the method further comprising mixing the hydrogel precursor with a photo-crosslinker before casting the hydrogel precursor in the mold.

In an embodiment, the hydrogel precursor is introduced into the mold in successive portions, with each portion being centrifuged at 4,500 rpm for 5 minutes prior to addition of the subsequent portion.

In an embodiment, the hydrogel precursor is introduced to the mold under light-free conditions until dry, and is then subjected to ultraviolet irradiation.

In an embodiment, the hydrogel precursor is exposed to ultraviolet irradiation at an intensity of about 400 μW/cm² for a period ranging from 5 to 20 minutes.

According to a second aspect of the present invention, there is provided a porous metallic microneedle device, comprising a substrate and one or more microneedles disposed on the substrate, wherein each microneedle is formed of a porous metallic material selected from the group consisting of silver, copper, gold, iron, and a combination thereof, the porous metallic material comprising interconnected pores having a pore size from about 1 nm to about 10 μm.

In an embodiment, each microneedle has a height of about 50 μm to 2000 μm.

In an embodiment, each microneedle has a base width of about 20 μm to about 300 μm.

In an embodiment, the microneedle is configured in a pyramid shape, a cone shape or a semi-pyramid shape.

In an embodiment, the device being made of silver, copper or alloy comprising silver or copper is antibacterial.

In an embodiment, the microneedles are arranged to form an array.

In an embodiment, the porous metallic microneedle device is configured to extract a body fluid selected from the group consisting of skin interstitial fluid, gingival crevicular fluid, gingival tissue fluid, gastrointestinal tract fluid, vascular fluid, and blood, wherein the device is optionally integrated with a catheter to facilitate sampling from the gastrointestinal tract or a blood vessel.

Embodiments of the present invention provide a method of fabricating a porous microneedle device in which the porous silver microneedles offer several key advantages. They are mechanically strong and easily penetrate skin tissue, enable rapid extraction driven by capillary force, and can capture a wide range of biomarkers-including small molecules, proteins, exosomes, and cells. The design also prevents retention of biomarkers during the subsequent release step. When fabricated from silver or copper or alloy containing silver or copper, the microneedles exhibit inherent antibacterial properties and resist blockage by tissue or cell debris.

### BRIEF DESCRIPTION OF THE FIGURES

Embodiments of the invention will now be described, by ways of examples only, with the accompanying figures in which:

FIG. 1A is a flow chart showing an embodiment of a method of fabrication of a porous metallic microneedle device, according to an embodiment of the present invention;

FIG. 1B is a schematic diagram showing an embodiment a method of fabrication of a porous metallic microneedle device, according to an embodiment of the present invention;

FIG. 1C is a graph showing relationship between the temperature against time during a sintering step of the method of fabrication as shown in FIG. 1B;

FIG. 1D is a photographic representation of a porous silver microneedle array on a scale graph paper, according to an embodiment of the present invention;

FIG. 2A is a graph showing Fourier Transform Infrared (FTIR) test results of methacrylated hyaluronic acid (MeHA) and MeHA absorbed with silver nitrate, during an embodiment of a fabrication method according to the present invention;

FIG. 2B shows a molecular formula of MeHA, which demonstrates binding sites of silver ions of MeHA;

FIG. 2C is an illustration showing morphological changes of microneedles through an embodiment of a fabrication method according to the present invention, with the scale bar=10 mm;

FIG. 2D is a scanning electron microscope (SEM) image of MeHA microneedles, according to an embodiment of the present invention, with the scale bar=500 μm;

FIG. 2E is an SEM image showing a porous structure of a silver microneedle, according to the present invention, with the scale bar=500 μm;

FIG. 2F shows an enlarged view of the silver microneedle of FIG. 2E under 1000× and 7000× magnification, further demonstrating the porous structure;

FIG. 3A shows elemental analysis of a porous silver microneedle array according to an embodiment of the present invention, using EDS and with a scale bar of 50 μm;

FIG. 3B shows resistance test results of a porous silver microneedle array path in three directions, according to an embodiment of the present invention;

FIG. 3C shows an illustration of optical coherence tomography imaging of a porous silver microneedle insertion in agarose hydrogel, according to an embodiment of the present invention;

FIG. 3D shows a cross-sectional image of a porous silver microneedle array inserting in agarose gel, according to an embodiment of the present invention;

FIG. 3E shows quantification of an insertion depth of a porous silver microneedle in 1% and 1.4% agarose gel according to an embodiment of the present invention;

FIG. 3F shows illustration of a thermogravimetric analysis of a porous silver microneedle array in a sintering process, according to an embodiment of the present invention;

FIG. 4A shows results of a mercury intrusion porosimetry analysis of pore size distribution of a porous silver microneedle, according to an embodiment of the present invention;

FIG. 4B shows quantification of liquid extraction in 1% and 1.4% agarose gel in a period of 60 s, according to an embodiment of the present invention;

FIG. 4C shows real-time imaging of silver microneedles absorbing 10%, 50%, and 100% fetal bovine serum, according to an embodiment of the present invention;

FIG. 4D shows real-time imaging of silver microneedles absorbing 10%, 50% and 100% glycerin, according to an embodiment of the present invention;

FIG. 4E is a graph showing viscosities of tested solutions of FIGS. 4C and 4D;

FIG. 4F is a graph showing extraction time of the tested solutions of FIGS. 4C and 4D by the silver microneedles;

FIG. 4G is a graph showing quantification of extracted volume of the test solutions of FIGS. 4C and 4D by the silver microneedles;

FIG. 4H is a graph showing relating between extracted volume and viscosity of the test solutions of FIGS. 4C and 4D;

FIG. 5A shows a photographic representation of a rat skin treated with Trypan Blue by a porous silver microneedle device, according to an embodiment of the present invention;

FIG. 5B shows a photographic representation of a porcine skin treated with Trypan Blue by a porous silver microneedle device, according to an embodiment of the present invention;

FIG. 5C shows a photographic representation of porcine gingival by a porous silver microneedle device, according to an embodiment of the present invention;

FIG. 5D shows a fresh rat skin treated with a porous silver microneedle device under 10N force, according to an embodiment of the present invention, with a scale bar of 500 μm;

FIG. 5E shows a fresh porcine skin treated with a porous silver microneedle device under 10N force, according to an embodiment of the present invention, with a scale bar of 500 μm;

FIG. 5F shows a fresh porcine gingival treated with a porous silver microneedle device under 10N force, according to an embodiment of the present invention, with a scale bar of 500 μm;

FIG. 5G is a graph showing evaluation of penetration ratio of porous silver microneedles into a rat skin under different pressures (Scale bar=2000 μm) and corresponding quantification, according to an embodiment of the present invention;

FIG. 5H is a graph showing evaluation of penetration ratio of porous silver microneedles into a porcine skin under different pressures (Scale bar=2000 μm) and corresponding quantification, according to an embodiment of the present invention;

FIG. 5I is a graph showing evaluation of penetration ratio of porous silver microneedles into a porcine gingival under different pressures (Scale bar=2000 μm) and corresponding quantification, according to an embodiment of the present invention;

FIG. 5J is a graph showing results of mechanical test of porous silver microneedles and MeHA microneedle, according to an embodiment of the present invention;

FIG. 6A shows images of agar plates inoculated with S. aureus and E. coli after co-culturing with 3 types of microneedle array patches for 24 hours;

FIG. 6B shows images of chocolate agar plates inoculated with Pg and Fn after co-culturing with 3 types of microneedle array patches for 48 hours;

FIG. 6C is a graph showing quantification of copies of S. aureus on the plates after co-culturing with the 3 types of microneedle array patches of FIGS. 6A and 6B;

FIG. 6D is a graph showing quantification of copies of E. coli on the plates after co-culturing with the 3 types of microneedle array patches of FIGS. 6A and 6B;

FIG. 6E is a graph showing quantification of copies of Pg on the plates after co-culturing with the 3 types of microneedle array patches of FIGS. 6A and 6B;

FIG. 6F is a graph showing quantification of copies of Fn on the plates after co-culturing with the 3 types of microneedle array patches of FIGS. 6A and 6B;

FIG. 6G is a graph showing results of a cytotoxicity test of a porous silver microneedle array, according to an embodiment of the present invention;

FIG. 7A is a diagram showing an embodiment of a porous silver microneedle device (right), for sampling at GcM in rat oral cavity (left), according to an embodiment of the present invention;

FIG. 7B is a diagram showing an embodiment of a porous silver microneedle device (right), for sampling at GtM in rat oral cavity (left), according to an embodiment of the present invention;

FIG. 7C is an illustration of the establishment of a rat periodontitis model and GCF extraction with either commercial test paper (GcP) or single row porous silver microneedle array (GcM), according to an embodiment of the present invention;

FIG. 7D shows graphs of enzyme-linked immunosorbent assay (ELISA) analysis of GCF extracted by GcP and GcM from healthy mice (negative, N) and periodontitis mice (positive, P), according to an embodiment of the present invention;

FIG. 7E shows graphs of ELISA analysis of GCF and GTF extracted by GcM and GtM respectively from healthy mice (negative, N) and periodontitis mice (positive, P), according to an embodiment of the present invention;

FIG. 8A is a photographic representation of a single row of a porous silver microneedle device fixed on a 3D-printed holder, according to an embodiment of the present invention;

FIG. 8B is a photographic representation of a subject's oral cavity before applying the porous silver microneedle device of FIG. 8A;

FIG. 8C is a photographic representation of the subject's oral cavity under application by the porous silver microneedle device of FIG. 8A;

FIG. 8D is a photographic representation of the subject's oral cavity after application by the porous silver microneedle device of FIG. 8A;

FIG. 8E shows changes in DBP and SBP immediately after and 7 days after the application of FIGS. 8B to 8D (above); and changes in body temperature immediately after and 7 days after the application of FIGS. 8B to 8D, with the absolute value of all data calculated;

FIG. 8F is a graph showing pulse rates of subjects before, immediately after, and 7 days after the application of FIGS. 8B to 8D;

FIG. 8G is a graph showing ELISA IL-1β in saliva(S), GCF obtained using a commercial paper strip (GcP) and a microneedle extraction (GcM);

FIG. 8H is a graph showing ELISA IL-1β in saliva(S), GCF obtained using a a microneedle extraction (GcM) and gingival tissue fluid obtained from microneedle (GtM);

FIG. 8I is a graph showing MMP8 in saliva(S), GCF obtained using a commercial paper strip (GcP) and a microneedle extraction (GcM);

FIG. 8J is a graph showing MMP8 in saliva(S), GCF obtained using a a microneedle extraction (GcM) and gingival tissue fluid obtained from microneedle (GtM);

FIG. 8K is a table of safety assessment immediately after the application of FIGS. 8B to 8D;

FIG. 8L is a table of safety assessment 7 days after the application of FIGS. 8B to 8D;

FIG. 8M is a table of verification of the integrity of the porous silver microneedle device after the application of FIGS. 8B to 8D;

FIG. 9 is a table showing sampling details during a clinical test, using an embodiment of a porous silver microneedle device according to the present invention; and

FIG. 10 is an illustration showing an embodiment of a fabrication method of a porous silver microneedle device, a periodontal diagnosis using such device, the anti-bacterial property of the device, and a microneedle-based sensor, according to the present invention.

### DETAILED DESCRIPTION OF THE EMBODIMENTS

Microneedles (MNs) are the emerging technology for sampling biological fluids such as blood and skin interstitial fluid (ISF). The fluid flows into porous/hollow MNs under capillary forces or is extracted due to the swelling of polymeric MNs. When integrated with sensors, they enable real-time monitoring of biomarkers in either qualitative or quantitative way, supporting applications in postoperative care and chronic disease management. Among a wide range of MN designs, porous MNs are especially attractive as they allow the extraction of whole blood/ISF and don't have clogging issues. In contrast, hollow MNs sometimes face the clogging issue while swelling MNs only extract small molecule components of blood/ISF. Although there is no report of using hollow or swelling MNs for GCF extraction, similar issues are expected as GCF contains proteins, cell derivatives and debris.

Porous MNs are generally made of biocompatible metals, ceramics, or polymers with small randomly distributed and interconnected pores. Metallic porous MNs are advantageous in term of their stronger mechanical strength and longer shelf life than polymeric and ceramic MNs. In the case of GCF extraction, metallic MNs do not crumple up under the pressure of insertion. This also minimizes the contact with blood and saliva, subsequently less contamination. However, metallic porous MNs are usually fabricated under high sintering temperatures (>1,000° C.) and strongly reducing atmosphere (e.g. hydrogen), which are both inconvenient and unsafe.

Under this circumstances, embodiments of the present invention provide a porous metallic MN device for the minimally invasive and contamination-free GCF sampling. The MN array in the device is made of antimicrobial silver and contain pores in the size range of 1 nm to 50 μm, and preferably from 1 nm to 10 μm, allowing the entrance of both biomolecules and cells. It is fabricated through sintering the silver-containing hydrogel MNs made of highly swellable methacrylated hyaluronic acid (MeHA) under 700° C. in the normal air atmosphere.

The MN array is then assembled with a 3D printed holder that facilitates the operation. In the rat periodontitis model, a 4×1 MNs array can extract 2~4 μL GCF within 30 s. In comparison, commercial test strip (2 mm×20 mm) can only extract 0.5~1 μL of fluid in 1 min. No bleeding was observed during the entire operation. The extracted GCF in MNs was collected through centrifugation and analyzed using ELISA to detect periodontitis related biomarkers including IL-1β qualitatively and quantitatively, IL-6, TGF-β1, VEGF, FGF, PGE2, and MMP-8. Compared with the commercial test strip group, concentrations of cytokines in the MN group were significantly higher, suggesting that there is less contamination and dilution of GCF by the saliva/blood in the MN group.

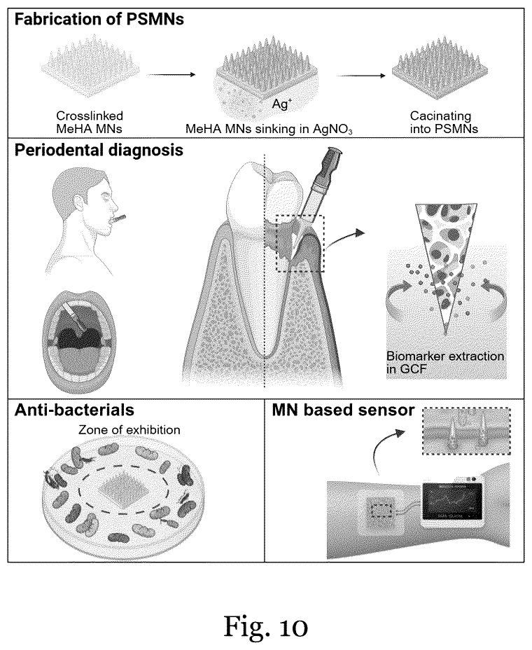

Accordingly, the local and systemic safety of the microneedle device of the present invention intended for the GCF collection is evaluated in periodontally healthy human subjects. The tissue reaction, infection, and allergy in the contact area at immediate post-application are examined without rest and 72 hours post-application. No sign of infection and/or allergy were observed. In terms of tissue reaction, there was also no bleeding, ulceration, edema, and erythema for all patients. Changes in systolic blood pressure (SBP), and diastolic blood pressure (DBP) for all subjects were within a clinically minimal range (Δ<15). The body temperature of all subjects was within an acceptable range. The pulse rates of the five subjects only fluctuated slightly before (Time 0) and after the experiment (Time 0.1) and 7 days later. Therefore, this technology provides a patient friendly and precise GCF collection that allows the accurate diagnosis and monitoring of periodontal diseases. FIG. 10 shows an overview of an embodiment of the present invention.

### Fabrication and Characterization of Porous Silver MN Array

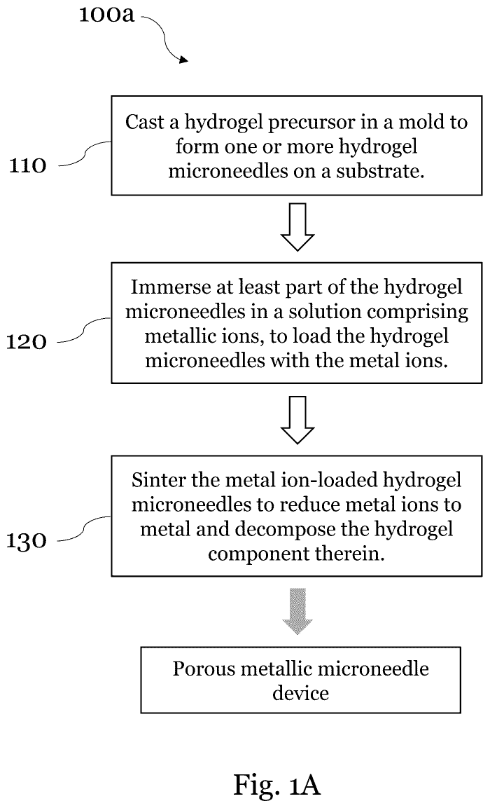

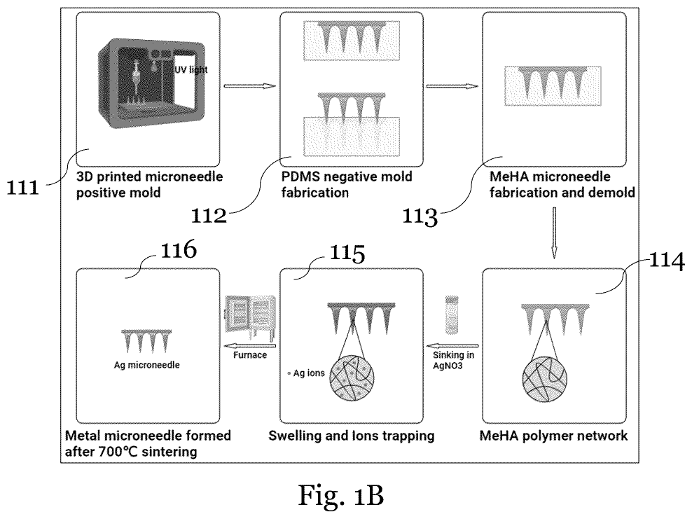

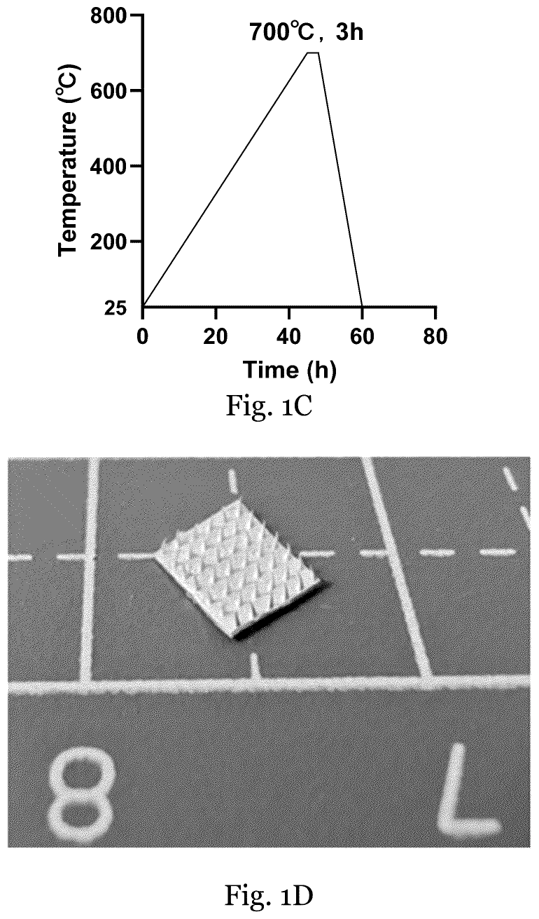

The porous silver MN array (PSMN) was fabricated through template-sacrificing method. The porous silver MNs were fabricated through template-sacrificing method, where MeHA MN patches swelled in the silver nitrate solution and formed hyaluronic silver complex. FIG. 1A shows an embodiment of a method 100a of fabricating a porous metallic microneedle device according to the present invention. The method 100 comprises step 110—casting a hydrogel precursor in a mold to form one or more hydrogel microneedles on a substrate; step 120—immersing at least part of the hydrogel microneedles in a solution comprising metallic ions, to load the hydrogel microneedles with the metal ions; and step 130—sintering the metal ion-loaded hydrogel microneedles, which causes the loaded metal ions to reduce to metal and the hydrogel component to decomposes, thereby forming the porous metallic microneedle device.

FIG. 1B is a schematic diagram illustrating an embodiment of a fabrication method 100b for producing a porous metallic microneedle according to the present invention. Prior to the casting step 113, the method 100b further includes forming a positive microneedle master mold 111, for example by three-dimensional (3D) printing or other suitable methods. The resulting positive master mold is then used to form a negative mold by casting polydimethylsiloxane (PDMS) or an equivalent material onto the positive master mold 112, thereby creating a mold suitable for casting the hydrogel microneedles. In this embodiment, the hydrogel precursor used for preparing hydrogel microneedles in step 113 is methacrylated hyaluronic acid (MeHA). Alternatively, it may be other photo-crosslinkable or hydrogel-forming polymers commonly employed in microneedle fabrication, such as gelatin methacryloyl (GelMA), poly(methyl vinyl ether-co-maleic acid) (PMVE/MA), polyvinyl alcohol (PVA), polyvinyl pyrrolidone (PVP), chitosan, silk fibroin, polyethylene glycol diacrylate (PEGDA), sodium alginate, or combinations thereof. In other embodiments, the hydrogel precursor comprises one or more carboxy group, including hyaluronic acid, collagen, alginate, PLGA, or a combination thereof.

After the casting step, the hydrogel microneedles form a crosslinked MeHA polymer network 114. The microneedles are then immersed in a silver nitrate solution 115, causing the hydrogel to swell and absorb silver ions. In step 116, the silver ion-loaded hydrogel microneedles are placed in a furnace and sintered at 700° C. in an air atmosphere, thereby reducing the silver ions to metallic silver and decomposing the hydrogel matrix to form the porous silver microneedle.

During sintering, silver ions were reduced into metallic silver by hyaluronic acid (HA), the reducing agent. MeHA polymer also acts as the stabilizer and matrix during this process. FIG. 1C shows a graph of the temperature versus time during the sintering process. The metal-loaded hydrogel microneedles were sintered by raising the temperature by 0.25° C./min until the maximum temperature was reached. This temperature was then maintained for three hours before cooling at 2° C./min until room temperature was reached. The MeHA polymer also acted as a stabilizer and matrix during this synthesis. It is also possible that silver nitrate breaks down directly into silver oxide at 250-440° C., which decomposes into silver and oxygen. As HA easily degrades at high temperatures, the resulting silver occupies the space left by the HA and forms silver MNs.

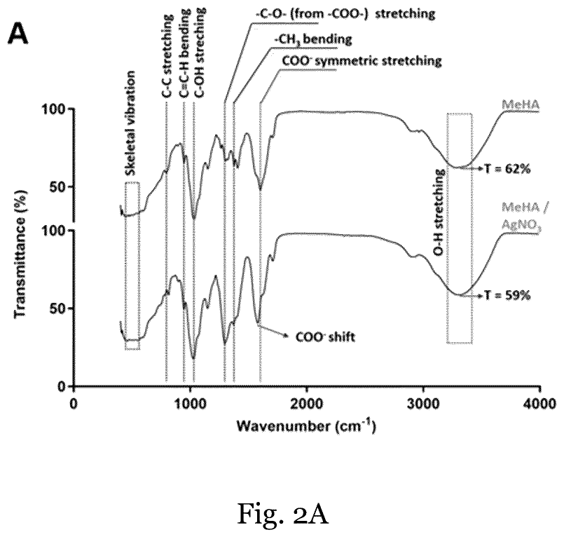

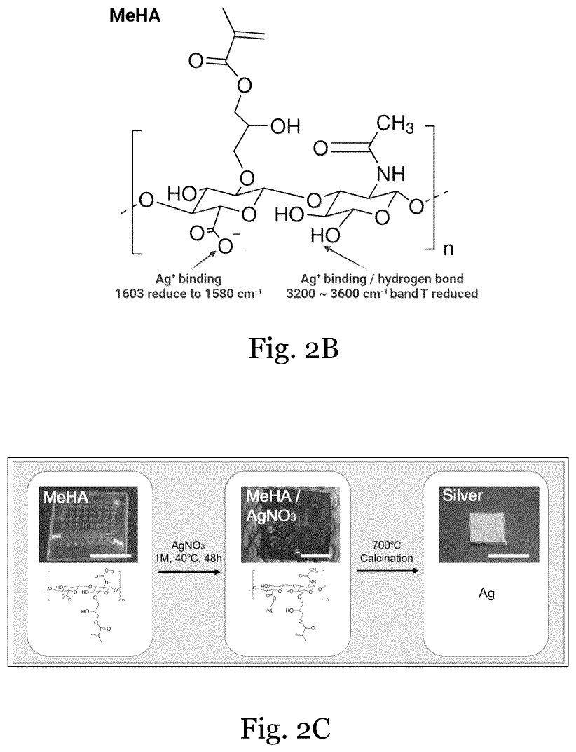

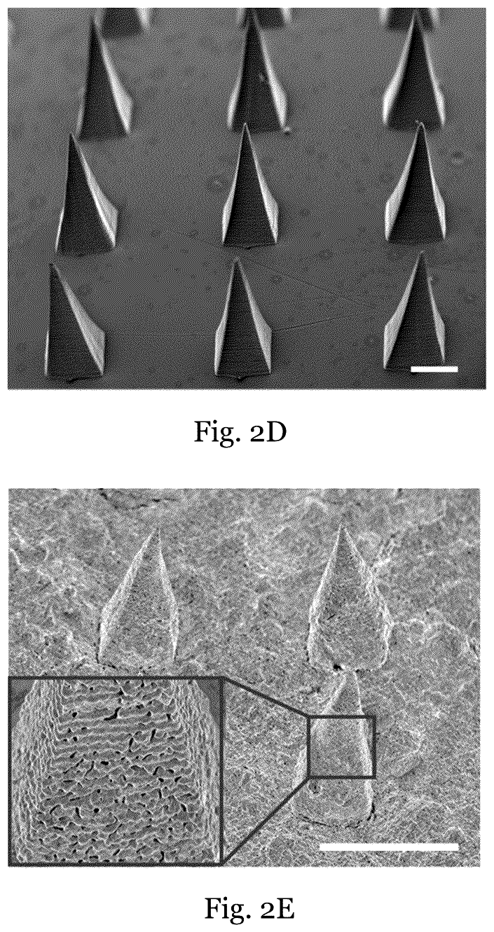

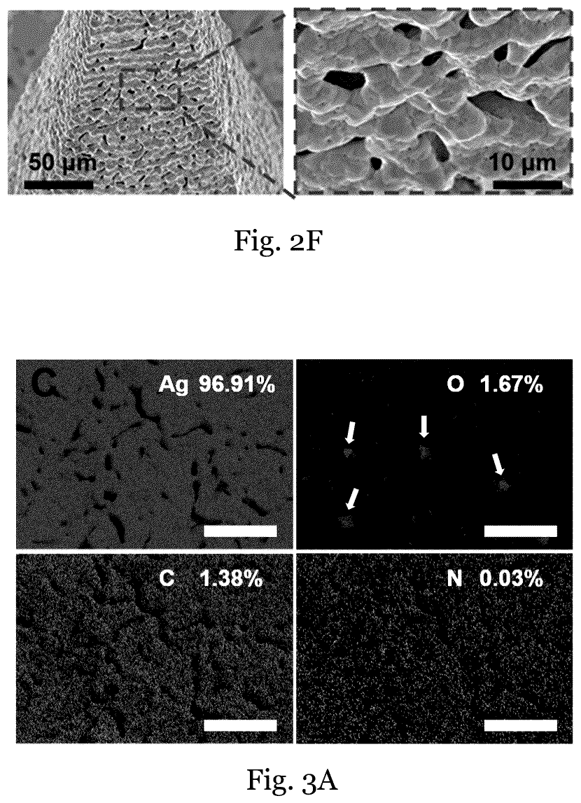

Referring to FIG. 2A, the fourier-transform infrared (FTIR) spectra of MeHA and MeHA/AgNO₃ reveal distinct chemical interactions between MeHA and silver nitrate in the range of 400-4000 cm. In the FTIR spectrum of MeHA, a broad O—H stretching vibration band was observed at 3200-3400 cm⁻¹. Upon formation of the hyaluronic acid-silver complex, the intensity of this O—H absorption band decreased (transmittance reduced from 62% to 59%), indicating a reduction in free hydroxyl groups due to coordination or hydrogen bonding between hydroxyl moieties (—OH) and Ag ions. Furthermore, a characteristic asymmetric stretching peak of the carboxylate group (COO⁻) was present at 1603 cm⁻¹ in the MeHA spectrum. Following interaction with silver ions, this peak shifted to 1580 cm⁻¹, suggesting coordination between COO groups and Ag⁺. This red shift is attributed to increased polarization of the C═O bond within the carboxylate group upon complexation. These spectral changes collectively confirm that both hydroxyl and carboxylate functional groups in MeHA are involved in binding with silver ions, forming a MeHA-Ag⁺ coordination complex. A molecular formula of which is shown in FIG. 2B, which shows the binding sites of silver ions on MeHA.

It is possible that silver nitrate directly broke down to silver oxide at 250-440° C. that decomposed into silver and oxygen. As HA easily degrades at high temperature, the derived silver occupied the space left by HA and formed the PSMN array. A photo representation of the PSMN array is shown in FIG. 1D. Thermogravimetric analysis (TGA) further confirms that the hydrogel gradually dehydrated as the furnace temperature increased from 25° C. to 180° C. With the temperature rose, MeHA burned in the air, and silver nitrate decomposed into silver oxide. During the process of reaching 600-700° C., the silver oxide gradually decomposed into silver.

During the entire sintering process, each MN array underwent approximately 50% shrinkage in size, accompanied by approximately 75% mass loss during the sintering process. FIG. 2C shows the morphological changes during the process. Originally, as shown in FIG. 2D, each MeHA MN tip had a height of 1.6 mm and a base of 0.6 mm. While the PSMN maintained the pyramid structure of MeHA MN, the length and diameter of MN tips became 500-600 μm and 5-10 μm respectively, as shown in FIG. 2D. Referring to FIG. 2F, the surface of the MN patch showed a visible porous structure with an average visible pore size on the needle surface of 1~10 μm. The derived silver atoms occupied the original space of degraded HA polymer.

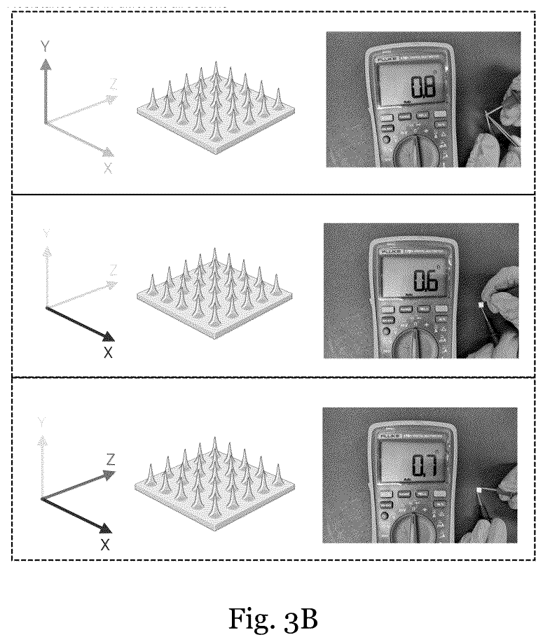

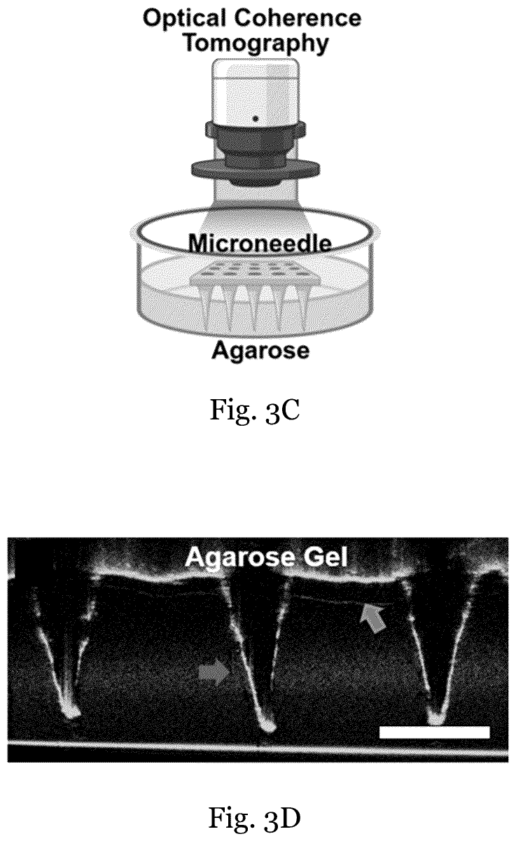

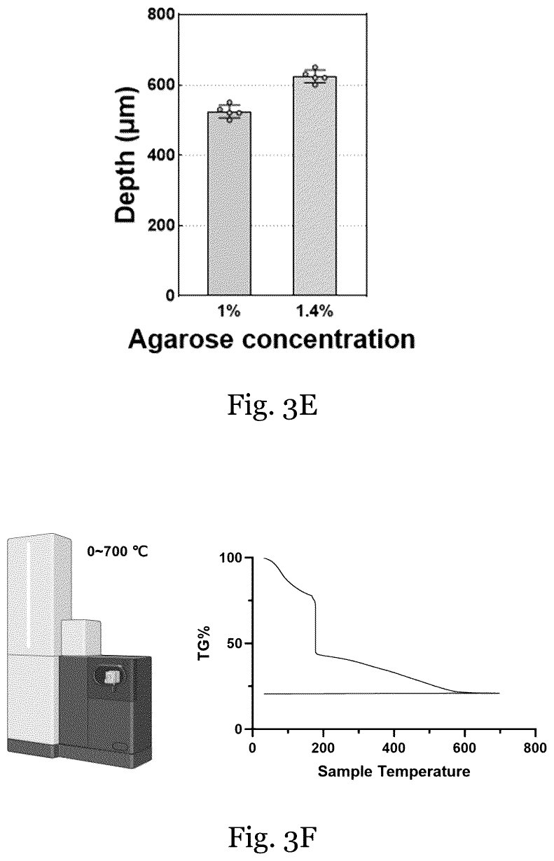

As explained, silver nitrate turned into silver either by the reduction of HA and/or the heat decomposition of silver nitrate. To confirm this, the chemical composition of the PSMN patch was examined by Energy-dispersive X-ray spectroscopy (EDS), as shown in FIG. 3A. There are mainly three elements: 96.91% (w/w) Ag, 1.67% O and 1.38% C. O is mainly distributed on some small protrusions on the surface of the sample, which are indicated by arrows on the figure. The main component of these small protrusions is silver oxide, which may be formed during sintering or storage. Due to the metallic nature, PSMNs shows exceptional electrical conductivity and minimal resistance. As shown in FIG. 3B, small resistance (0.6-0.8 ohms) was seen in all three different directions.

The metallic nature of PSMN enables visualization of its insertion process through optical coherence tomography (OCT). A linear scan, with the setup as shown in FIG. 3C, was carried to yield a cross-sectional image. Compared with agarose gel, metallic materials, as marked by the arrows in FIG. 3D, exhibit strong reflectivity in OCT imaging. Accordingly, the penetration depth in the 1% and 1.4% agarose gel can be quantified, which is shown in FIG. 3E.

The porous metallic microneedle device comprises a substrate with one or more microneedles disposed thereon. Each microneedle features interconnected pores having a pore size ranging from about 1 nm to about 50 μm, or preferably from about 1 nm to about 10 μm, while visible pores on the needle surface have a size ranging from 1 μm to 10 μm, enabling enhanced fluid wicking, drug loading, or extraction capabilities through capillary action while maintaining structural integrity.

In some embodiments, each microneedle has a height ranging from about 50 μm to about 2000 μm, which facilitates sufficient penetration through the stratum corneum into the viable epidermis or dermis for effective transdermal applications without causing significant pain or tissue damage. Also, each microneedle exhibits a base width ranging from about 200 μm to about 300 μm, providing a stable foundation on the substrate while ensuring mechanical robustness and precise skin insertion.

The microneedles may be configured in various shapes, including pyramid, cone, or semi-pyramid geometries, which optimize tip sharpness for reliable penetration and minimize insertion force requirements. In some embodiments, the device is made of porous metallic materials comprising silver, iron, copper, titanium, nickel, gold, chromium or manganese, or alloys comprising silver, iron, copper, titanium, nickel, gold, chromium or manganese, leveraging the antimicrobial properties of silver or the mechanical strength and biocompatibility of iron-based materials to support safe and effective use in biomedical contexts.

The microneedles are typically arranged to form an array, allowing for distributed delivery or sampling across a larger skin area and improving uniformity of performance.

In specific configurations, the porous metallic microneedle device is adapted to extract body fluids, such as skin interstitial fluid, gingival crevicular fluid, or gingival tissue fluid, by utilizing the interconnected porous network to draw fluids via passive capillarity for diagnostic or monitoring purposes.

### Liquid Extraction Capability of PSMN Array

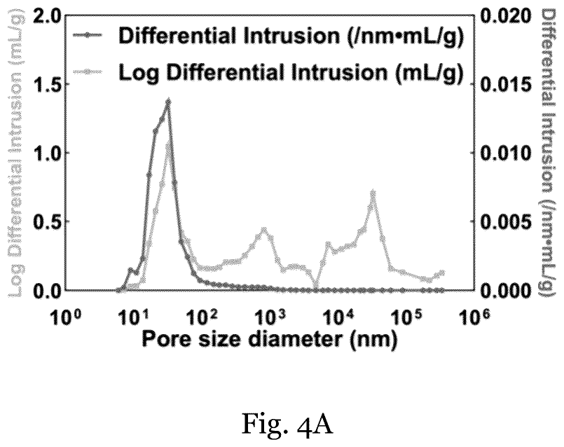

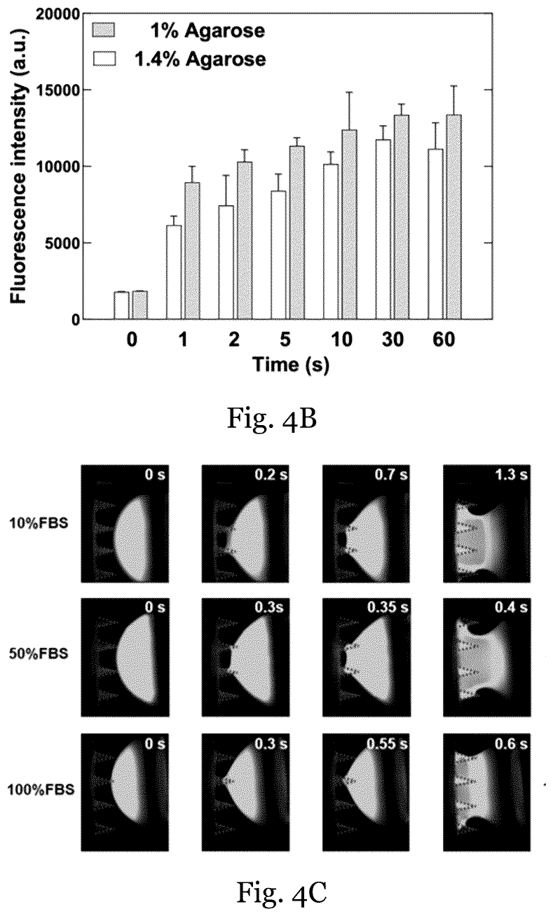

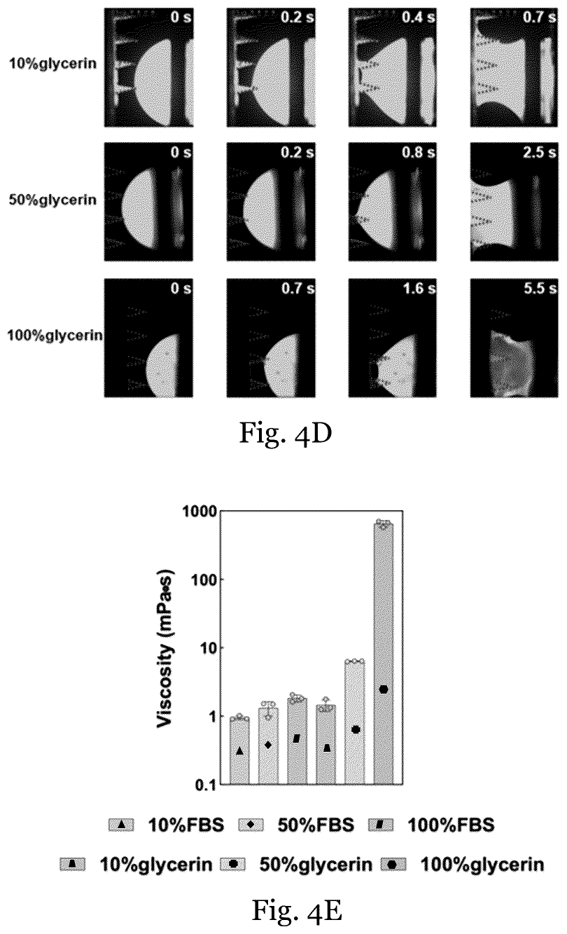

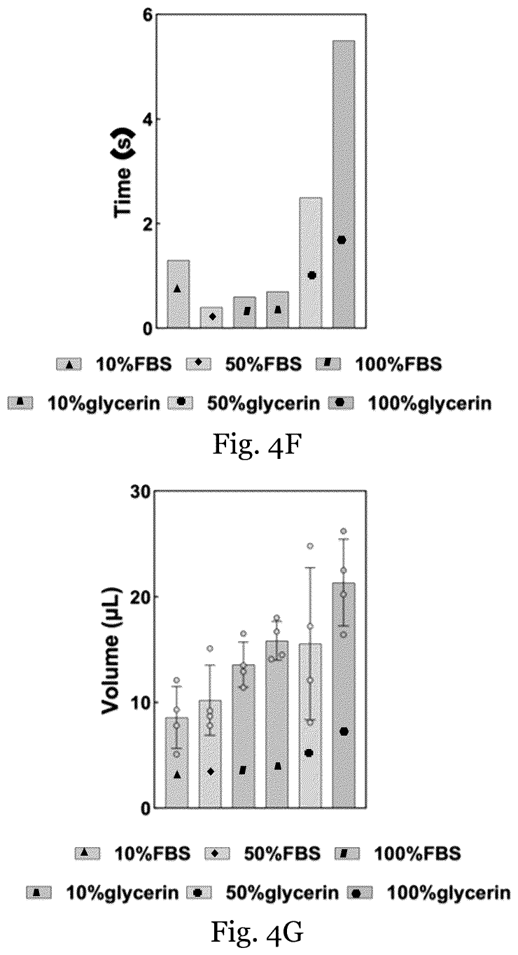

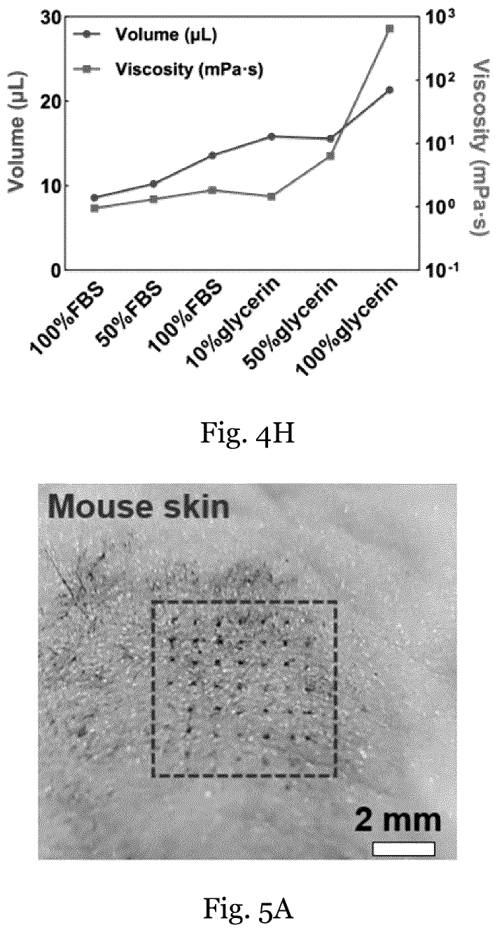

The most important property of the PSMN array is their porous structure. The pore size profile of which was quantitatively tested using the mercury intrusion porosimetry (MIP) method. FIG. 4A shows MIP analysis of the pore size distribution. The differential intrusion curve in FIG. 4A shows that pores in the range of 10-100 nm contribute most to the total porous volume. The log differential intrusion curve of FIG. 4A reveals that the sample contains pores from 10~ 102 nm and 104~ 105 nm in the entire pore size distribution profile.

Like other kinds of porous MNs, the porous structure provides the capability to extract liquid through the capillary force. For example, when PSMNs were placed in the agarose gels containing Fluorescein Isothiocyanate (FITC) dye, fluorescence from FITC was clearly visible in the porous MN tips within 30 s. The extraction speed of PSMNs was quantitatively examined, and results of which are shown in FIG. 4B. 1% and 1.4% agarose gels with 5 mg/ml FITC were used to simulate human soft tissue and skin, respectively. PSMNs were inserted into the gel for different times and were then placed in 1 ml of water to release the extracted fluorescent solution. The fluorescence of the water was measured, which reveals that extraction happened within is and reached the capacity at 10 s.

Throughout the body, biological fluids (saliva, blood, and synovial fluid etc) span orders of magnitudes of viscosity. This is due to the fluctuations in lipid and protein secretion and hydration levels. A series of solutions with different viscosities were prepared to fully access the liquid extraction capabilities of PSMNs. This includes 1×PBS, 10% FBS in 1×PBS, 100% FBS, 10% glycerol in 1×PBS, 50% glycerol in 1×PBS, and 100% glycerol. FIG. 4C shows real-time imaging of silver MNs absorbing FBS solutions, and FIG. 4D shows real-time imaging of silver MNs absorbing FBS solutions. The viscosity of these samples was measured using a viscometer, and in general, the higher the concentration of FBS and glycerol, the higher the viscosity, respectively, as shown in FIG. 4E. FITC was added to each solution (1 mg/ml) for fluorescence-based quantification purpose. PSMN tips were inserted into these solutions and the liquid migration along the MN tips was recorded using a camera. As shown in FIGS. 4C, 4D, and 4F, it only needed 0.6 s and 5.5 s to get the most viscous 100% FBS and 100% glycerol into the PSMN tips.

The liquid extracted by the MN array was further quantified by differential weight method (FIG. 4G) that measures the weight of liquids by differential gravimetry. The weight is then converted to volume by being divided by density. Within 10 s, the MN array extracted >8 μl in all solutions. We correlated the viscosity and extraction volume in FIG. 4H, where more liquid was extracted when the sample had a larger viscosity. This is because liquid with higher viscosity tends to adhere to the MN surface, resulting in a greater volume of solution being carried out. To note, PSMN array has with the size of 4 mm³ (μl) extracted >8 μl in all solutions, which suggests that PSMN is highly porous and able to extract more than 2 times its own volume. GCF is an inflammatory exudate derived from the periodontal tissues, composing serum and locally generated materials. Therefore, it is considered to have a similar viscosity to serum (100% FBS). Based on results in FIG. 4H, an 8×8 PSMNs could collect >10 μl GCF.

### Mechanical Properties of PSMN Tips

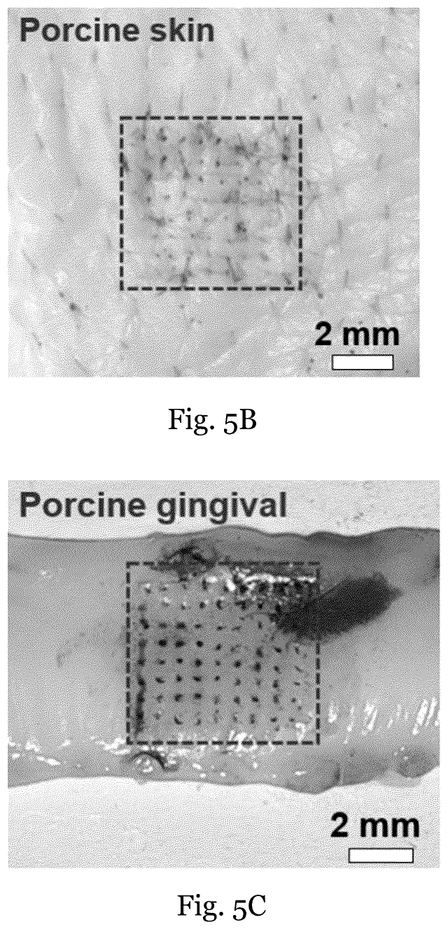

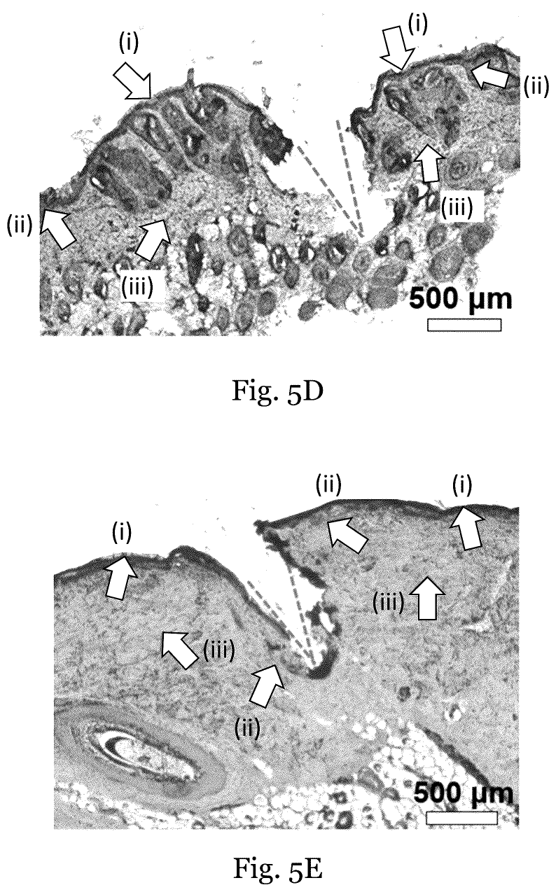

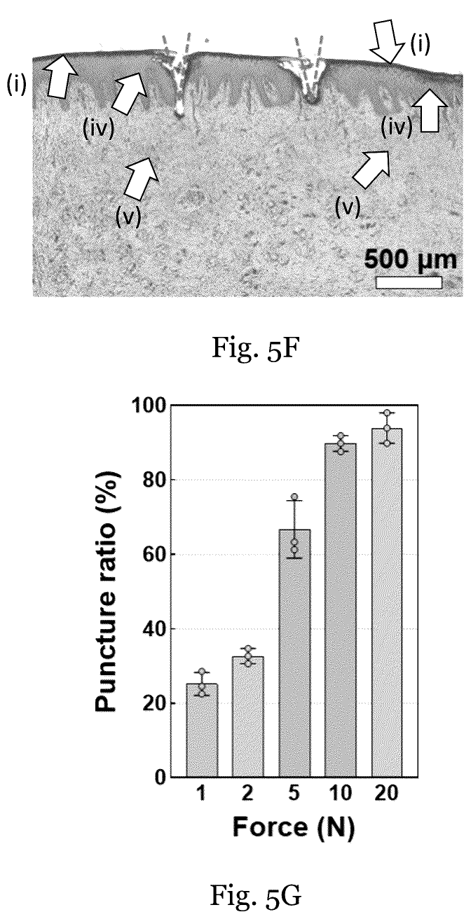

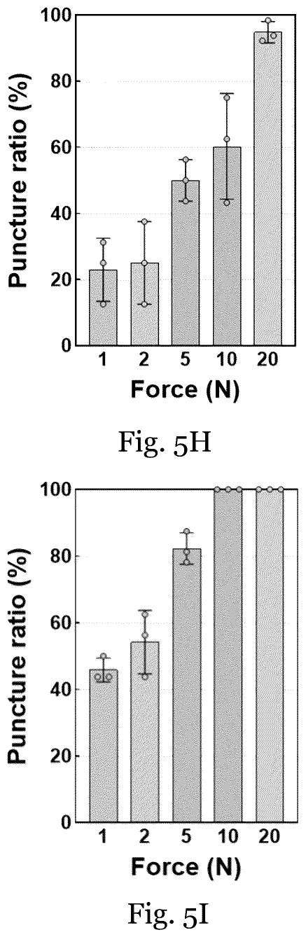

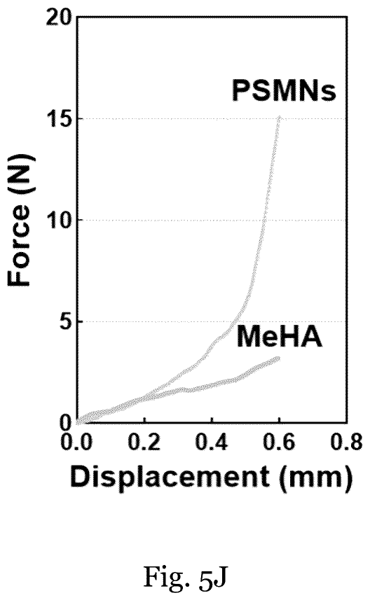

The penetration of the PSMNs was studied on the rat skin under different pressures (1, 2, 5, 10, 20 N). After insertion, PSMNs were removed, and skin was stained with Trypan blue. If the penetration was successful, there would be marks left, as demonstrated in FIG. 5A. The number of marks were counted to evaluate the success of skin penetration by PSMNs under different pressures. Results to which are shown in FIG. 5G. MNs achieved a penetration rates of approximately 25% under a 1 N force, 70% under a 5 N force and reached 90% under a 10 N force. A puncture test was conducted on porcine skin since the physical properties of porcine skin are closer to human, as shown in FIG. 5B. Rat and porcine skin treated by PSMNs under 10 N force was examined through histology (FIGS. 5D and 5E), where PSMNs easily pierced the stratum corneum and epidermis layers. MNs achieved a penetration rate of approximately 25% under a 1 N force, 50% under a 5 N force, and 95% under a 20 N force (FIG. 5H).

To prepare for subsequent gingival tissue fluid extraction experiments, fresh porcine gingiva was isolated and subjected to penetration testing, as shown in FIG. 5C. Histology of FIG. 5F shows that MNs readily pierced the gingival stratum corneum and reached the oral epithelium layer, in which the dotted line is the location of silver MNs, while the arrows in FIGS. 5D-5F represent (i) stratum corneum, (ii) epidermis, (iii) dermis, (iv) oral epithelium and (v) lamina propria. The MNs achieved penetration rates of approximately 45% under 1 N force, 85% under 5 N force, and 100% under 10 N force (FIG. 5I). From stained tissue sections, PSMNs were observed to penetrate approximately 600-700 μm into mouse and porcine skin, but only 400 μm into porcine gingiva. This is attributed to the Young's modulus of porcine and mouse skin being approximately 10 MPa, whereas that of porcine gingiva is 15-25 MPa. The higher mechanical strength of gingival tissue results in shallower penetration depth compared to skin tissue. Mechanical testing of PSMNs and MeHA MNs revealed that, under the same 0.6 mm compression condition, the loading force supported by the PSMN was 15.5 N, which was almost 5 times that of the MeHA MNs at 3.2 N, as shown in FIG. 5J.

### Antibacterial Property of PSMNs

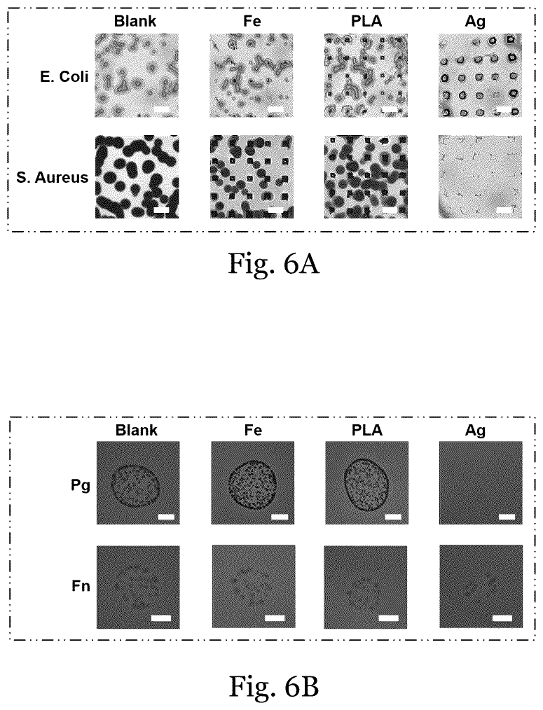

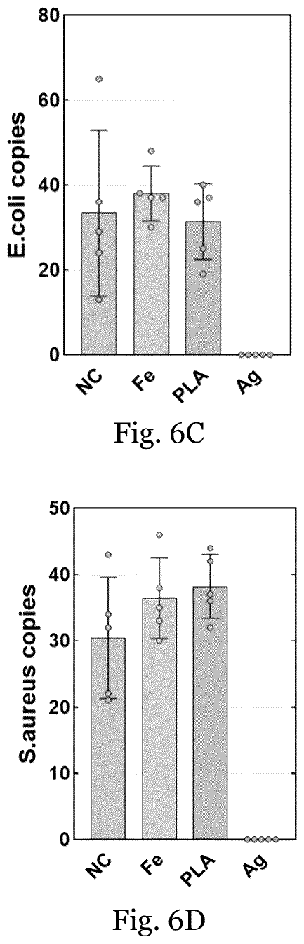

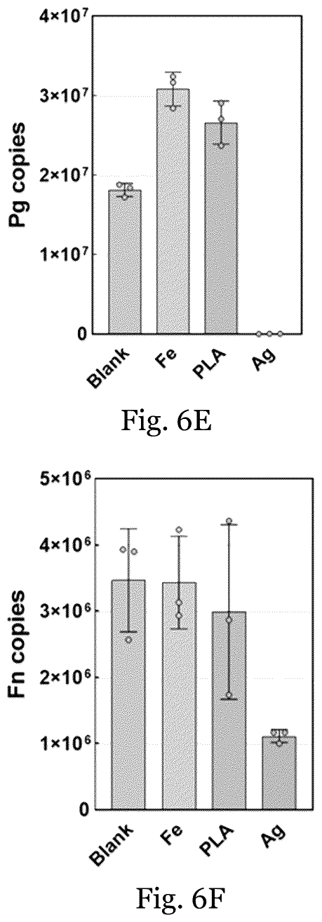

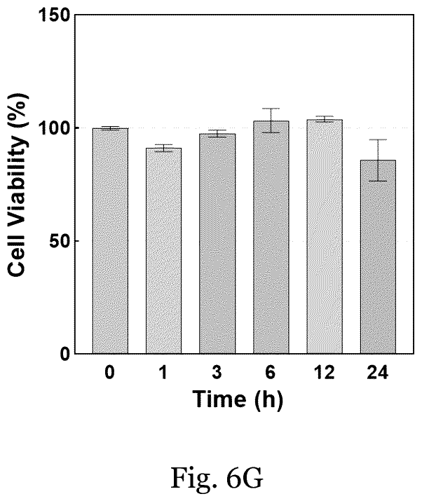

Silver has antibacterial property, which would minimize the introduction of any microbe into the sulcus/periodontal pocket during the GCF extraction. Referring to FIGS. 6A and 6B, the antibacterial properties of PSMNs (Ag) were compared with MNs made of stainless steel (Fe), polylactic acid (PLA), and blank group (NC). All MNs were sterilized by UV for 1 h before being placed on agar plates inoculated with gram-positive Staphylococcus aureus (S. aureus) and gram-negative Escherichia coli (E. coli). PSMN tips were inserted into the agar. The plates were incubated at 37° C. for 24 h. PSMNs completely eliminated both S. aureus and E. coli growth, as shown in the graphs of FIGS. 6C and 6D. In contrast, none of the other MNs inhibited the growth/proliferation of bacteria.

Antibacterial activity was further evaluated against two periodontal pathogens closely associated with periodontitis, Porphyromonas gingivalis (Pg) and Fusobacterium nucleatum (Fn), using the colony-forming unit (CFU) enumeration method for quantification. Standardized bacterial suspensions were aliquoted and incubated with three types of microneedles under anaerobic conditions at 37° C. for 24 h. After incubation, each sample was serially diluted in sterile phosphate-buffered saline (PBS) at 10\hat{ }3, 10\hat{ }5, and 10\hat{ }7. Subsequently, 10 μL of each dilution was spread onto the corresponding agar plates and cultured anaerobically at 37° C. until visible, countable colonies formed (72 h). Viable bacterial counts were determined by enumerating plates containing 15-300 colonies, and the results were expressed as CFU/mL. Notably, PSMNs completely eliminated Pg and exhibited stronger inhibitory activity against Fn than both the Fe MN and PLA MN groups, as shown in the graphs of FIGS. 6E and 6F. This confirms the antibacterial property of our PSMNs. In addition, a cytotoxicity test of PSMN array was conducted using human skin fibroblasts, results of which are shown in FIG. 6G. PSMN array was trimmed to appropriate shapes (3 mm×1 mm, 4 needles per array) and placed in each well for 1, 3, 6, 12, and 24 hours. The test results showed that the PSMNs were non-toxic to the cells in the co-culturing, demonstrating its biocompatibility.

### GCF and GTF Collection Using PSMNs in the Rat Periodontitis Model

The rat periodontitis model was induced via a combination of retentive ligature and repetitive injections of lipopolysaccharide. Two kinds of body fluid were of concern, including GCF and gingival tissue fluid (GTF). GTF refers to fluid within gingival tissues. When there is an inflammatory response, such as in periodontal disease, the permeability of blood vessels increases, leading to an increased flow of this fluid into the periodontal pocket, resulting in the formation of GCF. Analyzing both GTF and GCF can give a more complete picture of periodontal disease, as GCF reflects events in the sulcus while GTF reflects the state of the connective tissue. Further, some biomarkers, such as inflammatory mediators, may be present in different concentrations in each fluid. Analyzing both increases the chances of detecting a comprehensive profile of the disease state.

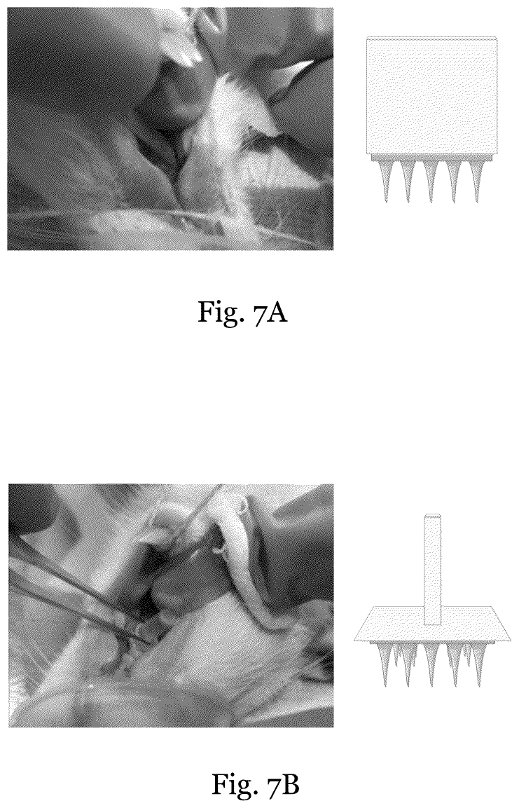

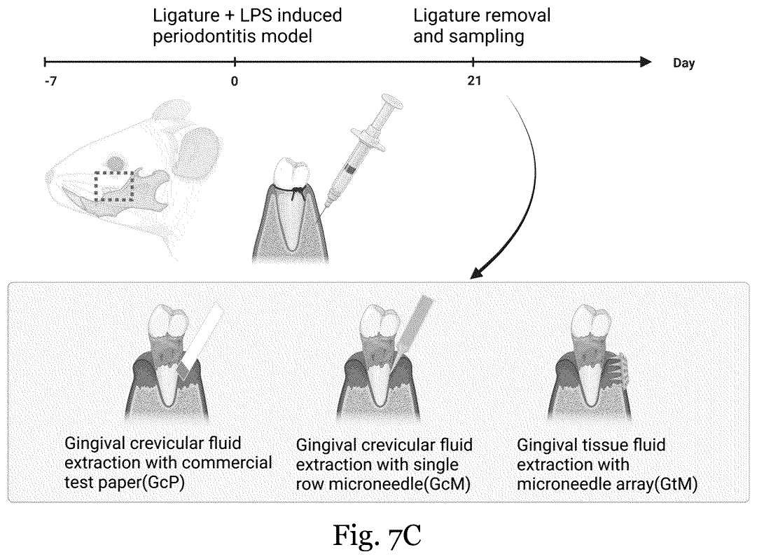

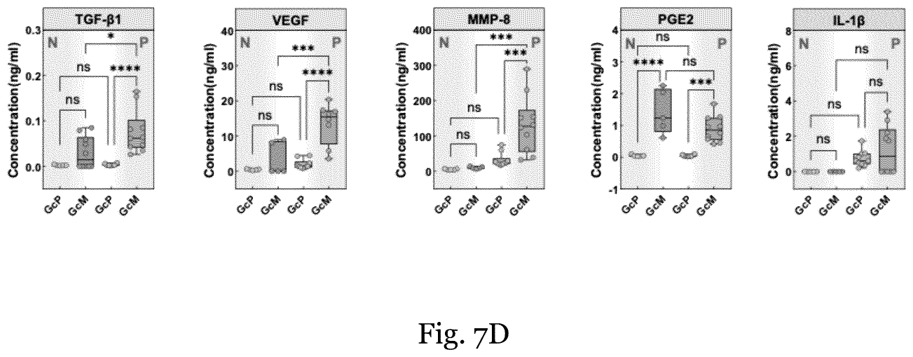

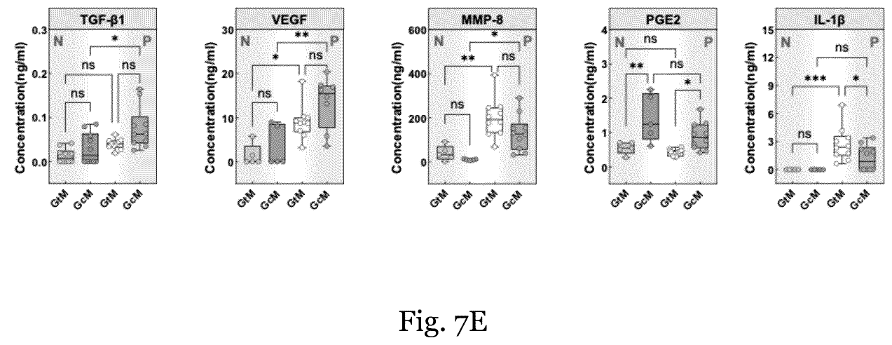

First of all, the GCF sampling were compared by inserting either commercial paper strip (GcP) or PSMNs (GcM) into the periodontal pocket. For easy comparison and convenience of insertion, GcM used single row PSMN patch (4 mm×1 mm×1 mm, 4×1 MNs per array), the same dimension as the commercial strip. The single row MN array was integrated with a 3D-printed holder to facilitate the sampling operations. An example to the single row MN array is shown in FIG. 7A (on the right). The extracted GCF and GTF were analyzed through enzyme-linked immunosorbent assay (ELISA) for the concentrations of TGF-β1, VEGF, MMP-8, PGE2, and IL-1β. As shown in FIG. 7D, the concentration means values of TGF-β 1, VEGF, MMP-8, PGE2 in the GcM are higher than those in the GcP for both healthy and diseased animals. The concentration mean value of IL-1β in the GcM is higher than that in the GcP for the diseased animals. This clearly indicates that there was less dilution of GCF by saliva when we used PSMN based devices for GCF sampling.

When GcM extracted biomarkers in the diseased group were compared with those in healthy groups, there are significantly higher levels of TGF-β1 (0.076±0.048 ng/ml vs 0.060±0.023 ng/ml), VEGF (13.51±5.88 ng/ml vs 3.42±4.69 ng/ml), MMP-8 (131.75±81.59 ng/ml vs 10.26±3.16 ng/ml) in the diseased animals. The elevated levels of these three biomarkers have been widely observed in both animal and human samples. For instance, a 2025 clinical study on paediatric patients confirmed that the levels of MMP-8 and TGF-β1 in GCF can be used to evaluate periodontal status. If the commercial paper strip (GcP) was used, there was no significant difference of TGF-β1, VEGF, and MMP-8 levels between the diseased group and the healthy groups. For PGE2 and IL-1β, there were no significant difference between samples from diseased rats and normal rats in both GcP and GcM groups. This analysis double confirms the reliability and precision of PSMN based devices for GCF sampling.

While GCF can clinically be sampled using paper strip, there is no method to sample GTF in the clinic. Therefore, there is barely any report to compare the contents of GCF and GTF. Here, for the first time, the PSMNs of an embodiment of the present invention enables the sampling of both GCF and GTF in a similar and minimally invasive way, GcM and GtM respectively. A schematic diagram of the sampling processing using the PSMNs is shown in FIG. 7C. Different from GcM (a single row PSMN, 4 mm×1 mm×1 mm, 4 MNs), GtM has four rows of PSMN (4 mm×4 mm×1 mm, 4×4 MNs). But both were integrated with a 3D-printed holder to facilitate the sampling operations (illustrations on the right of FIGS. 7A and 7B).

As shown in FIGS. 7D and 7E, in the GcM samples, there are significantly higher levels of VEGF, MMP-8 and no significant difference of PGE2 concentration between samples from diseased and normal rats. Here similarly, in the GtM group, there are significantly higher levels of VEGF (9.02±3.87 ng/ml vs 1.44±2.48 ng/ml), MMP-8 (200.12±88.62 ng/ml vs 39.59±33.68 ng/ml) in the diseased animals than the normal animals, as shown in FIG. 7D. And there was no difference of of PGE2 concentrations between diseased rats and normal rats in the GtM group as well. However, the results of TGF-β1 and IL-1β are different in the GtM group, as shown in FIG. 7E. There was significantly higher level of TGF-β1 in the diseased animals if GCF was used for analysis. This difference disappeared when GTF was used. In terms of IL-1β, we didn't observe the significantly elevated IL-1β level in the diseased animals in GCF sample. However, GTF sample from disease animals showed significantly higher level of IL-1β that is related to the progression of periodontal disease.

Based on the results in FIGS. 7D and 7E, it is concluded that PSMN provides a reliable and precise method to sample GCF with less dilution by saliva, enabling the accurate diagnosis of periodontal disease from the elevated expression of different biomarkers. GTF can be extracted using PSMNs and provide additional data to verify the results based on GCF. The absence of significant PGE2 differences in rat periodontitis model is known. The induction of PGE2 requires simultaneous exposure to oral pathogens and inflammatory cytokines. If the disease model lacks sustained cytokine-pathogen synergy, PGE2 may not reach detectable thresholds. Furthermore, PGE2 exhibits pulsatile secretion, and single-timepoint measurements may miss peak concentrations.

### Observational Clinical Safety Study

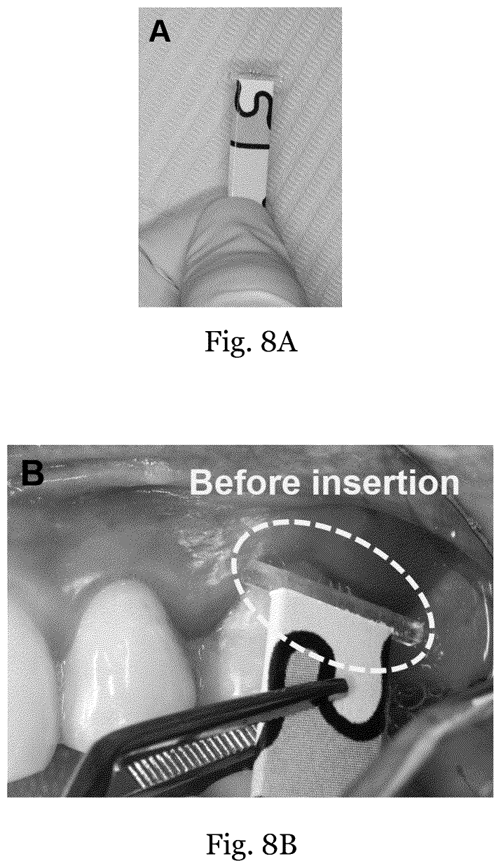

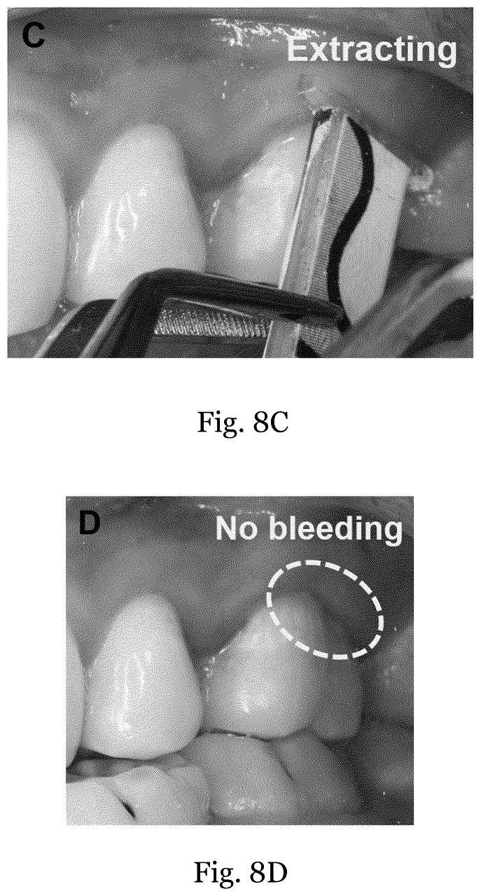

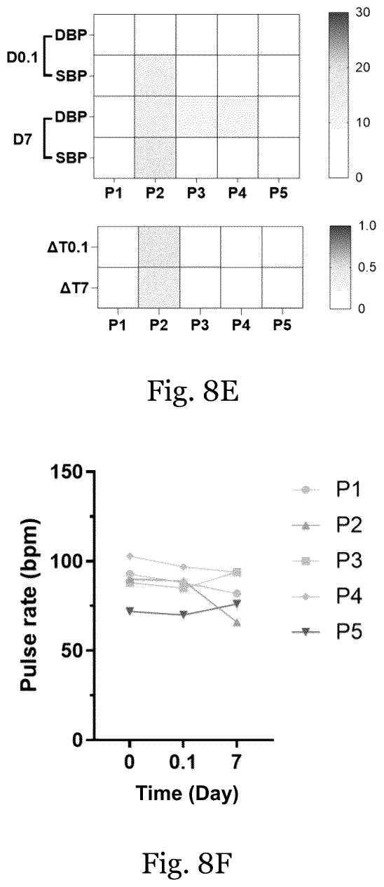

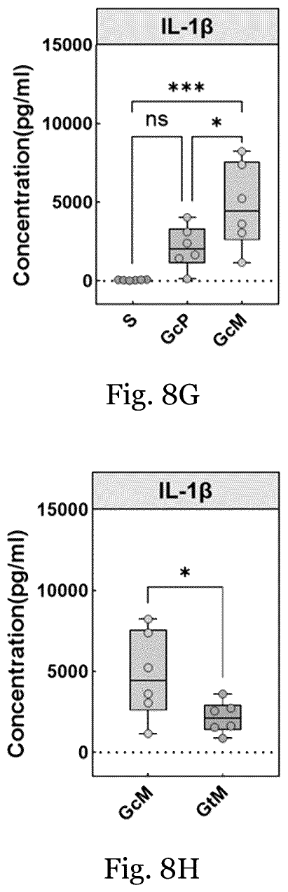

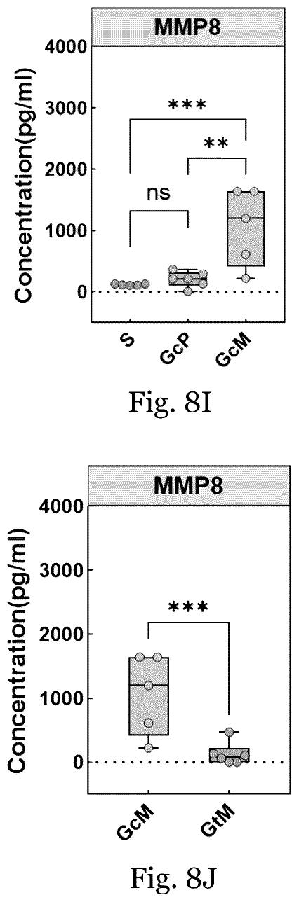

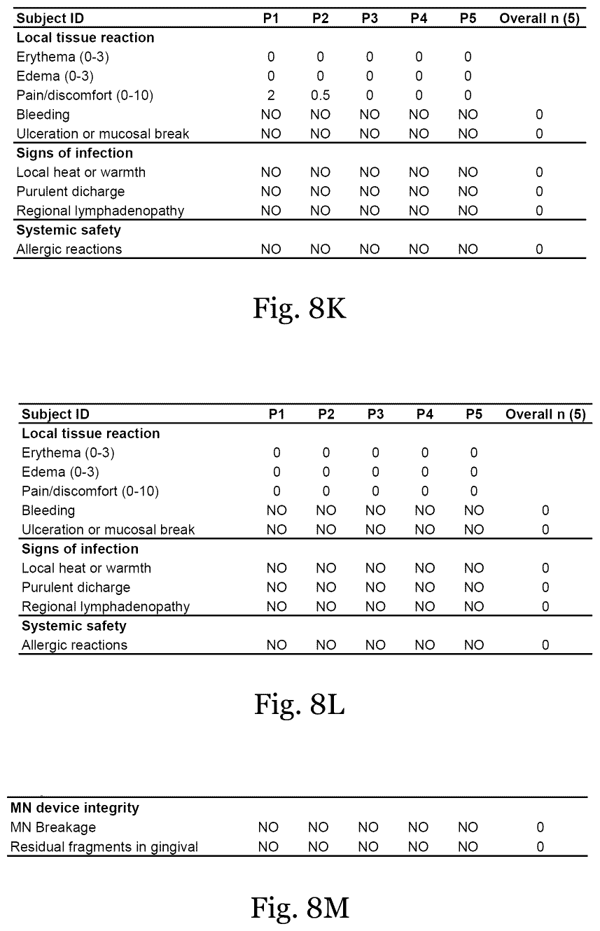

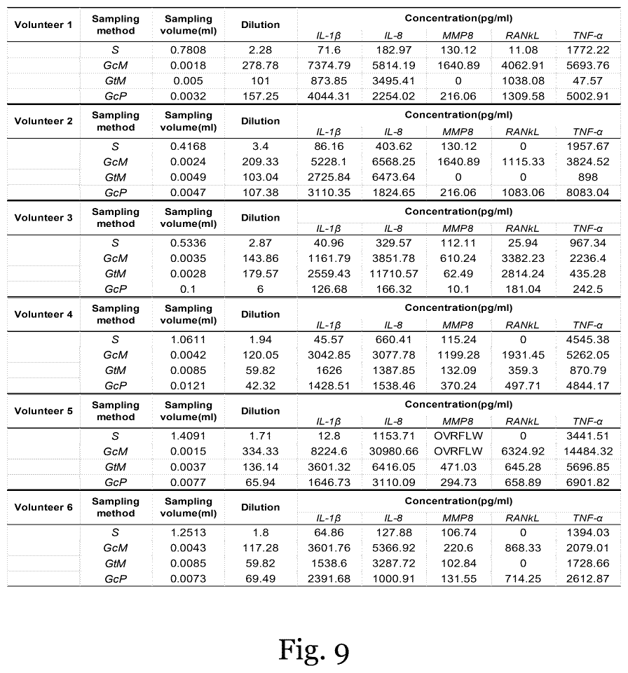

The local and systemic safety of this GcM device intended for the GCF collection in periodontally healthy human subjects, an example of which is shown in FIG. 8A, was evaluated. It is a prospective, single-arm, observational clinical safety study. The sterile GcM device was inserted into the bottom of gingival sulcus at mesial buccal site of each first molar for 30 s. No MN facture was observed in all patients (FIGS. 8B and 8K). The tissue reaction, infection, and allergy in the GcM contact area at immediate post-application without rest and 72 hours post-application are also evaluated (FIGS. 8B, 8C, 8K, 8L). No sign of infection and/or allergy were observed. In terms of tissue reaction, there was no bleeding (FIG. 8D), ulceration, edema, and erythema for all patients (see the table of FIG. 8K). However, 2 out of 5 patients reported slight pain during the sampling (see the table of FIG. 8L).

Systolic blood pressure (SBP), and diastolic blood pressure (DBP), pulse and body temperature of volunteers were also monitored. SBP and DBP are primary indicators of cardiovascular load and systemic vascular resistance. A significant increase could suggest elevated cardiac afterload or vasoconstriction, while a sharp decrease might indicate circulatory failure. Their stability is fundamental to systemic safety. In a healthy individual, the normal range of SBP fluctuations in a day is usually within 20-40 mmHg, and the normal range of DBP fluctuations in a day is within 10-20 mmHg. FIG. 8E shows that changes in SBP and DBP for all subjects were within a clinically minimal range (Δ<15). The body temperature of all subjects varied within 0.5 degrees Celsius, which is within an acceptable range (normal body temperature variation in a day is 1° C.).

Pulse rate (PR) is related to the health condition of cardiovascular system.37 Under normal circumstances, the pulse rate of adults fluctuates widely throughout the day but generally falls between 60 and 100 beats per minute (bpm). FIG. 8F shows that the pulse rates of the five subjects fluctuated slightly before (Time 0) and after the experiment (Time 0.1) and 7 days later. However, the overall distribution was between 50 and 100 bpm. Among them, P4's baseline pulse rate was 103 bpm, slightly above 100 bpm. This finding provides strong evidence for the systemic safety of the intervention from a cardiovascular homeostasis perspective.

IL-1β and MMP-8 are quantified in samples collected from volunteers, including saliva(S), GCF obtained using a commercial paper strip (GcP) or microneedle extraction (GcM), and gingival tissue fluid obtained from microneedle (GtM). Concentrations of both IL-1β (FIG. 8G) and MMP-8 (FIG. 8I) in GCF collected using PSMNs (GcM) were higher than those obtained using commercial paper strips (GcP) and saliva(S). IL-1β in the GcM group reached 4772.32±2735.6 μg/mL, whereas the GcP group showed 2125.71±1411.39 μg/mL. MMP8 concentration in the GcM group was 1062.38±628.79 μg/mL while it was 206.46±126.31 μg/mL in GcP group. These suggest that PSMNs enabled collection of GCF with significantly higher IL-1β and MMP8 levels than those measured in GCF obtained by paper strips. Both cytokines were present at very low concentrations in saliva.

The concentrations of IL-1β and MMP8 in gingival tissue fluid obtained from microneedle (GtM) are also compared with GcM. As shown in FIG. 8H, IL-1β levels in GTF (GtM, 2454.17±1009.61 μg/mL) were also significantly lower than those in the GcM group, indicating that IL-1β is more enriched in GCF while remaining detectable in gingival tissue. Similar phenomenon was observed in MMP8 detection, as shown in FIG. 8J. Concentrations were 1062.38±628.79 μg/mL (GcM) and 128.08±175.76 pg/mL (GtM) respectively.

### Clinical Data

A total of 6 subjects participated in the experiment, aged 25 to 50 years, with no gender restrictions. All subjects agreed to have full-mouth periapical radiographs taken and signed an informed consent form. 30 minutes before the clinical examination, subjects rinsed their mouths with water to remove food debris. All subjects underwent 4 collection tests, with a collection interval of 10 minutes. Before and after collection, all samples were weighed and recorded, and the weight of the extracted liquid was calculated using the different method, assuming a liquid density of 1 g/cm³. All samples were stored in EP tubes and placed in a cooler or dry ice box throughout the collection process, and then transferred to −20° C. for storage. During the experiment, the procedures for the four experimental groups are as follows.
1. Saliva collection group(S): Collect 0.5-1 ml of naturally excreted saliva from the patients and place it in a 5 ml EP tube. Before and after collection, weigh and record the EP tube, and add 2 ml of TE buffer.
2. Test strip collection of gingival crevicular fluid group (GcP): Use commercial filter paper strips (Whatman®, 3 #, size: 4 mm×40 mm). Collect from 4 locations, which are the gingival crevices of the outer side of the first and second premolars. Insert the strip and keep it in place for 1 minute. Each person uses 4 filter paper strips for 4 separate collections. After collection, place the 4 filter paper strips together in a 2 ml EP tube and add 1 ml of TE buffer.
3. Microneedle collection of gingival crevicular fluid group (GcM): Collect gingival crevicular fluid from the gingival crevices of the outer side of the first and second premolars for 30 seconds. Each person uses 4 separate samples for 4 collections. After collection, remove the microneedle samples used for the 4 collections with forceps and place them in a 2 ml EP tube. Add 1 ml of TE buffer.
4. Microneedle collection of gingival tissue fluid group (GtM): Press the microneedle sample onto the gingival surface of the outer side of the second premolar for 30 seconds. Each person uses 2 separate samples for 2 collections. After collection, remove the front part of the 2 samples of microneedles using forceps and place them in a 2 ml EP tube. Add 1 ml of TE buffer.

After completing all 4 collections, subjects rested for 30 minutes and were allowed to leave if they did not experience any discomfort. The collected samples were analyzed for IL-1β, IL-6, IL-8, TNF-α, MMP8, and RANKL levels using an ELISA kit (Elabscience®) method. Results of the clinical test are shown in table 9.

Embodiments of the invention provide a porous metallic microneedle device and a method of fabrication thereof, to enable reliable and precise GCF sampling in the diagnosis of periodontal disease. The MN array was fabricated through template-sacrificing method, where MeHA acted as the template and silver took the space after the reduction from silver ions. The surface of PSMN patch showed a visible porous structure with an average pore size of 1~10 μm, which permitted the extraction of liquids with a wide range of viscosity in less than 10 s. The highly porous structure also allowed the extraction of more than 2 times its own volume. The silver property provided not only good mechanical strength to enter periodontal pockets without deformation but the anti-bacterial property to avoid the potential contamination of oral bacterial. In the periodontitis rat model, in comparison with the clinically used paper strips, PSMNs show faster extraction of GCF, minimal dilution by saliva and less retention of three cytokine biomarkers (TGF-β1, VEGF, and MMP-8). This leads to more accurate diagnosis of periodontitis in the animal models. It also permitted the sampling of GTF that provided supportive information in the diagnosis. Finally, we confirm the safety of this device through a prospective, single-arm, observational clinical safety study, in which there is no tissue reaction, infection, or allergy in all volunteers. As PSMNs are electrically conductive, we will integrate biosensors onto the device that can realize the conversion of biomarker concentrations to the digital signals.

Embodiments of this invention can help realize a reliable and precise sampling of GCF for the accurate assessment of the periodontal conditions in the clinical settings. Determination of the stage and grade of periodontitis in the rat model is based on the gingival sulcus bleeding index (GSBI), gingival index (GI), and probing depth. The model is successful when:
1. The rat gingival tissue is swollen and there is slight bleeding on probing (GSBI≥3);
2. Obvious gingival inflammation (GI≥2);
3. The probing depth is increased compared with the normal group, which is statistically significant.
(1) Gingival sulcus bleeding index: Use a periodontal probe to gently probe the periodontal pocket or gingival pocket at the modeling site for 10 seconds, and score according to the following:
0=healthy gingival margin and gingival papilla, no bleeding after probing.
1=mild inflammation of the gingival margin and gingival papilla, no bleeding after probing.
2=mild inflammation of the gingival margin and gingival papilla, gingival color changes, no edema, and punctate bleeding after probing.
3=moderate inflammation of the gingival margin and gingival papilla, gingival color changes, mild edema, bleeding after probing, and spreading along the gingival margin.
4=moderate inflammation of the gingival margin and gingival papilla, gingival color changes, moderate edema, bleeding after probing, overflowing the gingival margin.
5=moderate inflammation of the gingival margin and gingival papilla, gingival color changes, severe edema, ulceration, and spontaneous bleeding tendency after probing.
(2) Gingival index: The measurement site and expression method are the same as the gingival sulcus bleeding index. The animal experiment operator observed and recorded the macroscopic indicators with the naked eye:
0=Normal gingiva.
1=Mild inflammation, slight color change, no bleeding on probing.
2=Moderate inflammation, redness, edema, bleeding on probing.
3=Severe inflammation, obvious redness, edema, ulceration, and a tendency to spontaneous bleeding.
(3) Probing depth: Record the distance from the gingival margin to the periodontal pocket or the bottom of the gingival pocket. The deepest point of the periodontal pocket (or gingival sulcus) is recorded as the maximum probing depth. Six measurement sites are selected from the mesial to distal side of the palatal side of the first molar, and a total of 12 sites on the left and right sides are averaged to represent the probing depth of the sample.

Study design for safety evaluation of microneedle (MN) device for Gingival Crevicular Fluid (GCF) collection
1. Type: Prospective, single-arm, observational clinical safety study
2. Participants: 5 adult volunteers
Inclusion criteria:
   Periodontal health subjects according to 2017 classification [1]
   Age 18-65 years
   Good general and oral health (no active periodontal disease or oral mucosal lesions)
   No allergy history to device materials
   Non-smokers
   Willingness to provide informed consent and comply with study procedures
Exclusion criteria:
   Pregnancy or breastfeeding
   Edentulism
   Use of medications impairing healing (e.g., immunosuppressants, anticoagulants)
   Habits that may compromise oral mucosa integrity
   Uncontrolled systemic diseases
3. Duration: Single MN application with follow-up at immediate post-application without rest and 72 hours post-application
4. Study Procedure
4.1) Baseline Assessment:
   Vital signs recording: systolic blood pressure (SBP), and diastolic blood pressure (DBP), body temperature, and pulse rate
   Periodontal examination and clinical diagnoses: one single trained and calibrated examiner will perform the full-mouth periodontal examination consisting of the following measurements.
1) the position of the gingival margin (GM) in relation to the cementoenamel junction (CEJ) at six sites (mesio-buccal, buccal, disto-buccal, mesio-lingual, lingual, disto-lingual) per tooth;
2) probing pocket depth (PPD) at six sites (mesio-buccal, buccal, disto-buccal, mesio-lingual, lingual, disto-lingual) per tooth;
3) bleeding on probing (BOP) at six sites (mesio-buccal, buccal, disto-buccal, mesio-lingual, lingual, disto-lingual) per tooth and full-mouth bleeding score (FMBS);
4) clinical attachment level (CAL, calculated as PPD+GM) at six sites (mesio-buccal, buccal, disto-buccal, mesio-lingual, lingual, disto-lingual) per tooth;
5) furcation involvement (FI) according to the Hamp's classification
6) the degree of tooth mobility according to Miller (1950);
7) the number of tooth loss due to periodontitis.
4.2) PSMN patch application:
   1) Use sterile tweezers, carefully insert a Microneedle strip into the bottom of gingival sulcus at mesial buccal site of each first molar.
   2) After 30 seconds, the Microneedle strip will be removed.
4.3) Immediate Post-Application Assessment without rest: Inspection for bleeding, erythema, edema, pain assessment
5. Follow-up Assessments: At 72 h, post-application for safety evaluation

### Safety Assessment Parameters and Outcomes

| | | | |
|---|---|---|---|
| | Assessment | | |
| Parameter | Method | Timing | Outcome Measures |

Local tissue reaction

| | | | |
|---|---|---|---|
| Erythema | Visual | Immediate | Graded by Modified |
| (redness) | inspection, | without | Draize |
| | standardized | rest, 72 h | Scale: 0 (none) |
| | photography | | to 3 (severe) |
| Edema | Visual and | Immediate | Graded 0-3 |
| (swelling) | tactile | without | (none to severe) |
| | assessment | rest, 72 h | |
| Bleeding | Visual | Immediate | Presence/absence; |
| | observation | without | duration measured |
| | | rest, 72 h | in minutes |
| Pain/ | Subject self- | Immediate | VAS score 0 |
| Discomfort | report via | without | (no pain) to 10 |
| | Visual Analog | rest, 72 h | (worst pain) |
| | Scale (VAS) | | |
| Ulceration | Visual | Immediate | Presence/absence; |
| or mucosal | inspection | without | lesion size |
| break | | rest, 72 h | (mm) if present |

Signs of Infection

| | | | |
|---|---|---|---|
| Local heat | Palpation | Immediate | Presence/absence |
| or warmth | | without | |
| | | rest, 72 h | |
| Purulent | Visual | Immediate | Presence/absence |
| discharge | inspection | without | |
| | | rest, 72 h | |
| Regional | Palpation of | Immediate | Presence/absence |
| lymphadenopathy | regional | without | |
| | lymph nodes | rest, 72 h | |

Systemic Safety

| | | | |
|---|---|---|---|
| Vital signs | Standard | Baseline, | Any clinically |
| (Blood pressure, | clinical | immediate | significant deviation |
| pulse) | instruments | without | from baseline |
| | | rest, 72 h | |
| Allergic | Observation | Immediate | Rash, urticaria, |
| reactions | and subject | without | anaphylaxis |
| | report | rest, 72 h | (presence/absence) |

MN Device Integrity

| | | | |
|---|---|---|---|
| MN breakage | Visual | Immediate | Presence/absence of |
| | inspection | without | broken microneedle |
| | post-use | rest | fragments |
| Residual | Visual | Immediate | Presence/absence |
| fragments | inspection, | without | |
| in gingiva | radiography | rest | |
| | if needed | | |

### Material and Method

Methacrylated hyaluronic acid (MeHA, Q-HA09100) was purchased from Kamulin Biotech (Xi'An, China). Silver nitrate (AgNO₃≥99.99) was from Henan Yuheng Chemical Co., Ltd. (Zhengzhou, Henan). Agarose (low EEO, ST004L) was acquired from Beyotime Biotechnology Inc (China). Polydimethylsiloxane (PDMS, Dow Corning 184 Sylgard) were purchased from Dow®. AlamarBlue cell viability reagent, LIVE/DEAD™ viability/cytotoxicity kit and CellTracker™ Green CMFDA were purchased from Thermo Fisher Scientific (USA). All ELISA kits were purchased from ElabscienceR. All other materials except specifically mentioned were acquired from Sigma-Aldrich (Singapore).

Fabrication of PSMNs: Firstly, an 8×8 MN array master resin mold was fabricated through 3D printing (Prismlab Co. Ltd., China). The dimension is 2 cm×2 cm×0.2 cm. Each MN has a quadrangular pyramid shape with a height of 1.6 mm and a base of 0.6 mm. The PDMS negative mold was made using the reported protocol.

MeHA solution (20 mg/ml) was then mixed with photocrosslinker (Irgacure 2959, w/w=100:1) in the dark for 12 hours. And 0.8 ml of the mixture was then added to the PDMS mold. The mold was subject to centrifugation at 4,500 rpm for 5 min before another 1.4 ml mixture was added. The sample was left in the dark until dried. Later, another 1.4 mL mixture was added and dried in the dark. This was repeated three times until the total amount of MeHA solution reached 6.4 ml. MeHA MN patch was subsequently irradiated under 400 uW/cm2 UV for 5, 10, and 20 min respectively to obtain MNs with different crosslinking degrees.

Finally, MeHA MNs were placed in the silver nitrate solution (0.25, 0.5, 1, 2, 4 M) at 40° C. After 48 h, the surface of MNs was wiped using filter paper to remove excess silver nitrate solution before they were put into a muffle furnace (KSL-1100X-S, Shenzhen Kejing Star Technology, China). The sintering was carried with a 0.25° C./min ramp rate and a maximum temperature of 700° C. in the air atmosphere. After 3 h at 700° C., the sample was cooled at 2° C./min to room temperature.

Morphology of MNs: Morphology of MNs was examined by Scanning Electron Microscope (FEI Quanta 250 Environmental SEM). The frozen sections were observed and photographed under a Nikon Ti2-A fluorescence Microscope.

Cytotoxicity test of PSMNs: Human skin fibroblasts (NF127, CellResearch Corp Pte Ltd, Singapore) were cultured (37° C., 5% CO2) for 3 days before cytotoxicity test. Cells were then seeded in a 96-well plate, with 5 replicates per group. PSMNs were sterilized by UV for 1 h before the test. MNs were trimmed to proper shapes (3 mm×1 mm, 4 needles per array) and placed to each well for 1, 3, 6, 12, and 24 hrs. Later, PSMNs were removed and the pre-mixed alamar blue reagent was added to the wells. After 1-hour incubation, cells were rinsed with PBS before examination using the microplate reader (SpectraMax® M5e, Molecular Devices).

Antibacterial testing: The antibacterial ability of MNs were studied using an agar diffusion assay.39 Specifically, gram-positive S. aureus and gram-negative E. coli were grown overnight in an incubator shaker at 37° C. The bacterial cell suspension was centrifuged at 4,000 rpm for 5 min. The bacterial cell pellet was then resuspended in 1× phosphate buffered saline and diluted until the optical density at 600 nm was approximately 0.004. S. aureus and E. coli were inoculated onto agar plates using a sterile swab. MN patch was sterilized by UV for 1 hour before they were placed on the inoculated plates with the needle being inserted into the agar. The inoculated plates were incubated at 37° C. for 24 hours. Afterwards, the MN patch was removed, and optical images were taken to determine the extent of bacterial growth. Each assay was repeated 5 times and quantified using Image J.

Periodontitis rat model: SD rats (male, 150-250 g) were provided by Zhuhai Baishitong Biotechnology Co., Ltd. with the experimental animal production license number of SCXK (Guangdong) #2020-0051. The experimental procedure was approved by the Institutional Animal Care and Use Committee of Guangdong Lewwin Pharmaceutical Research Institute Co., Ltd. (#IA-CMA-2023031-01) and the Animal Research Ethics Subcommittee of the City University of Hong Kong (AN-STA-00000389).

Five rats were reserved as the negative control group. Other ten rats were anaesthetized by the intraperitoneal injection of tiletamine hydrochloride and zolazepam hydrochloride (Virbac Corp, TX, USA) at the dosage of 25-75 mg/kg. A periodontal probe was used to measure the periodontal pocket depth of the second molars on both the left and right sides of the upper and lower jaws. These measurements were recorded, and the gingival status was checked and photographed. Dental floss was used to increase the distance between the first and second molars. A gingival separator was employed to separate the gingiva of the second molar, fully exposing the tooth neck. During the separation process, 0.5% lidocaine (Jilin Huamu Animal Health Product Co. Ltd, China) was applied locally for infiltration anesthesia. A 4-0 silk thread was tied around the tooth neck in the gingival sulcus, and a buccal knot was secured. The tail end of the ligature was pressed into the gums with a dental filler. Meloxicam was administered subcutaneously for analgesia for at least three consecutive days after the silk thread ligation, with an initial dose of 0.2 mg/kg and a maintenance dose of 0.1 mg/kg.

After the ligation was completed, 1 μL of 10 mg/mL lipopolysaccharide (LPS, 039M4004V) was injected into the gingiva of the second molar. And this was repeated 2 times per week at each site. Before each injection, 2-3% isoflurane (Hebei Jindafu Pharmaceutical Co. Ltd, China) was inhaled for anesthesia, and there was at least 1-day gap between injections. After the model was successfully established (criteria were listed in Supporting Information), a maintenance dose of 20 μg/mL LPS was injected every day. The rats were given 10% sugar water in the morning and switched to drinking pure water in the afternoon.

Extraction of oral cavity samples in rats: We collected three fluids in the rat model including gingival crevicular fluid extracted with commercial test paper (GcP), Gingival crevicular fluid extracted with MN (GcM), Gingival tissue fluid extracted with MN (GtM). Before operation, both the MN device and commercial test strip were sterilized by UV. Each device was weighed before and after the sampling to calculate the amount of collected liquid. The tooth crown was cleaned with cotton rolls before sampling to eliminate any potential interference.

GcM extraction was carried using a long strip-shaped 3D printed handle with a fixed MN array (4×1) at one end. The MNs were inserted into the oral pouch of the rat's second molar for 30 seconds and then placed in 100 μl of TE buffer and stored at −20° C. Extract 2 times from each rat and combine the 2 samples for testing.

GcP was performed similarly. The test strip was inserted into the oral pouch of the rat's second molar for 30 seconds. Then placed in 100 μl of TE buffer and stored at −20° C. Since the amount of gingival crevicular fluid obtained from the test strip is minimal, use 4 test strips for 4 extractions from each rat and combine them for testing.

In GtM extraction, a 4×4 MN array was fixed on a hammer-shaped 3D printed handle. The device was pressed on the gum tissue of the inner side of the rat's second molar for 30 seconds, then placed in 100 μl of TE buffer and stored at −20° C.

Safety evaluation in volunteers: The test was carried on 5 adult volunteers (18-65 years ago) with good general and oral health (no active periodontal disease or oral mucosal lesions). GcM MN tips were inserted into the bottom of gingival sulcus at mesial buccal site of each first molar. After 30 seconds, the GcM device was removed. We examined local tissue reactions, signs of infection, systemic safety and MN device integrity immediately and 72 hours after MN device removal. Details are included in the supplementary information.

The porous metallic microneedle device is designed to extract body fluids for disease diagnosis and monitoring of therapeutic efficacy. Body fluids include gingival tissue fluid, gingival crevicular fluid, and skin interstitial fluid. The body fluid extracted using this device contains small molecules, biomolecules, exosomes and cells. The body fluid can be released from the device through centrifuge or dilution using buffer before analysis using cell sequencing, mass spectrometry, ELISA, and proteomics etc. In an embodiment, wherein the microneedle device is integrated with a catheter to facilitate sampling from the gastrointestinal tract or a blood vessel

In comparison with clinically used devices (paper strip) or other microneedle devices such as swellable microneedles and hollow microneedles, the porous silver microneedles have the following advantages: 1) mechanically strong and penetrate into the skin tissue easily; 2) fast extraction due to the capillary force; 3) extract a wide range of biomarkers including small molecules, proteins, exosomes and cells; 4) no retention of biomarkers during the following releasing step; 5) antibacterial when it is made of silver or copper, or alloys containing silver or copper; 6) no blockage by the tissues/cell debris.

In term of gingival crevicular fluid (GCF) exaction, currently, GCF is mainly collected using test strip (cellulose paper). In both animal and human test (data is attached in manuscript and ppt slides), we found that our microneedle device can extract more GCF. In comparison to the commercial test strip group, a 4 mm×20 mm commercial test strip can extract about 0.5~1 μl of fluid in 1 min. The concentrations of cytokines in the MN group were significantly higher, which suggests that there is less contamination and dilution of GCF by the saliva in the MN group.

The collection and analysis of gingival crevicular fluid (GCF) is a recognized approach to develop an accurate picture of periodontal diseases and treatment effectiveness. This article proposes and demonstrates the use of porous silver microneedles (MNs) to extract GCF. Metallic porous MNs are advantageous in terms of stronger mechanical strength and longer shelf life than polymeric and ceramic MNs. Different from conventional fabrication of metallic MNs using high temperature sintering (>1,000° C.) under a strongly reducing atmosphere (e.g. hydrogen), we develop a new way (hydrogel-guided fabrication) to make the porous silver MNs under relatively low temperature (700° C.) in the normal air atmosphere. The derived MNs can easily penetrate through fresh tissues and extract 10 μL interstitial fluid in less than 10 s through the capillary force. The silver property also inhibits bacterial growth around the MN patch. In the periodontitis rat model, in comparison with the clinically used paper strips, silver porous MNs show faster extraction, less contamination from saliva and less retention of cytokine biomarkers in the extracted fluid.

The invention has been given by way of example only, and various other modifications of and/or alterations to the described embodiment may be made by persons skilled in the art without departing from the scope of the invention as specified in the appended claims.

1. A method of fabricating a porous metallic microneedle device, comprising steps of:
casting a hydrogel precursor in a mold to form one or more hydrogel microneedles;
immersing at least part of the hydrogel microneedles in a solution comprising metallic ions, to load the hydrogel microneedles with the metal ions; and
sintering the metal ion-loaded hydrogel microneedles to form the porous metallic microneedle device.

2. The method as claimed in claim 1, wherein the sintering of the metal-loaded hydrogel microneedles is performed at a maximum temperature of about 700° C. in an air atmosphere.

3. The method as claimed in claim 2, wherein the metal-loaded hydrogel microneedles are sintered by raising the temperature at 0.25° C./min to the maximum temperature, maintaining at the maximum temperature for a predetermined period, and then cooling at 2° C./min to room temperature.

4. The method as claimed in claim 3, wherein the wherein the metal-loaded hydrogel microneedles are maintained at the maximum temperature for about 3 hours.

5. The method as claimed in claim 1, wherein the hydrogel precursor comprises one or more carboxy group, wherein the hydrogel precursor is selected from a group consisting of hyaluronic acid, collagen, alginate, PLGA, or a combination thereof.

6. The method as claimed in claim 1, wherein the metallic ions are silver ions or iron ions.

7. The method as claimed in claim 6, wherein the metallic ions are silver ions and the solution is a silver nitrate solution.

8. The method as claimed in claim 7, wherein the silver nitrate solution has a concentration ranging from 0.25M to 4M.

9. The method as claimed in claim 1, further comprising mixing the hydrogel precursor with a photo-crosslinker before casting the hydrogel precursor in the mold.

10. The method of claim 1, wherein the hydrogel precursor is introduced into the mold in successive portions, with each portion being centrifuged at 4,500 rpm for 5 minutes prior to addition of the subsequent portion.

11. The method as claimed in claim 10, wherein the hydrogel precursor is introduced to the mold under light-free conditions until dry, and is then subjected to ultraviolet irradiation.

12. The method as claimed in claim 11, wherein the hydrogel precursor is exposed to ultraviolet irradiation at an intensity of about 400 μW/cm² for a period ranging from 5 to 20 minutes.

13. A porous metallic microneedle device, comprising a substrate and one or more microneedles disposed on the substrate, wherein each microneedle is formed of a porous metallic material selected from the group consisting of silver, copper, gold, iron, and a combination thereof, the porous metallic material comprising interconnected pores having a pore size from about 1 nm to about 10 μm.

14. The porous metallic microneedle device as claimed in claim 13, wherein each microneedle has a height of about 50 μm to 2000 μm.

15. The porous metallic microneedle device as claimed in claim 13, wherein each microneedle has a base width of about 200 μm to about 300 μm.

16. The porous metallic microneedle device as claimed in claim 13, wherein the microneedle is configured in a pyramid shape, a cone shape or a semi-pyramid shape.

17. The porous metallic microneedle device as claimed in claim 13, wherein the device being made of silver, copper or alloys comprising silver or copper is antibacterial.

18. The porous metallic microneedle device as claimed in claim 13, wherein the microneedles are arranged to form an array.

19. The porous metallic microneedle device as claimed in claim 13, configured to extract a body fluid selected from the group consisting of skin interstitial fluid, gingival crevicular fluid, and gingival tissue fluid.
